(19) United States
(12) Patent Application Publication
(10) Pub. No.: US 2011/0300111 A1
(43) Pub. Date: Dec. 8, 2011

(54) GENERATION OF INDUCED PLURIPOTENT STEM CELLS WITHOUT THE USE OF VIRAL VECTORS

(75) Inventors: Anthony J White, Melbourne (AU); Raj R Makkar, Los Angeles, CA (US); Eduardo Marbán, Beverly Hills, CA (US)

(73) Assignee: CEDARS-SINAI MEDICAL CENTER, Los Angeles, CA (US)

(21) Appl. No.: 13/130,560

(22) PCT Filed: Nov. 19, 2009
(86) PCT No.: PCT/US09/65117

§ 371 Date: Aug. 19, 2011

Related U.S. Application Data
(60) Provisional application No. 61/116,623, filed on Nov. 20, 2008.

(51) Int. Cl. A61K 35/34; A61P 9/04; A61P 9/10; C12N 5/071; A61P 9/00

(52) U.S. Cl. 424/93.7; 435/377; 435/325

(57) ABSTRACT

Presented herein, generally, are methods for generating reprogrammed mammalian cells, e.g., induced pluripotent stem cells, from differentiated mammalian cells without the use of viral or plasmid vectors. In one aspect, the methods involve contacting a differentiated cell with transducible polypeptides comprising a reprogramming factor polypeptide linked to a cell penetration peptide so that a reprogrammed mammalian cell that exhibits at least one characteristic of pluripotency is generated. Also presented herein are methods for cardiac differentiation of a mammalian cell without the use of viral or plasmid vectors. In one aspect, such methods involve contacting a mammalian cell exhibiting at least one characteristic of pluripotency with a transducible polypeptide, so that cardiac differentiation of the cell occurs.

### 1. CROSS-REFERENCE

The instant application claims the benefit of U.S. provisional application No. 61/116,623, filed Nov. 20, 2008, the disclosure of which is incorporated herein by reference in its entirety.

### 2. FIELD

Presented herein, generally, are methods for generating reprogrammed mammalian cells, e.g., induced pluripotent stem cells, from differentiated mammalian somatic cells without the use of viral or plasmid vectors. In one aspect, the methods involve contacting a differentiated somatic cell with transducible polypeptides comprising a reprogramming factor polypeptide linked to a cell penetration peptide so that a reprogrammed mammalian cell that exhibits at least one characteristic of pluripotency is generated. Also presented herein are methods for cardiac differentiation of a mammalian cell without the use of viral or plasmid vectors. In one aspect, such methods involve contacting a mammalian cell exhibiting at least one characteristic of pluripotency with a transducible polypeptide so that cardiac differentiation of the cell occurs.

### 3. BACKGROUND

The reprogramming of adult cells into a less differentiated state, e.g., into pluripotent stem cells, is of great medical importance. For example, pluripotent cells generated from readily available adult somatic cells (e.g., skin), may be utilized to make replacement cells for the treatment of diseased adult organs. Furthermore, creation of disease-specific cell lines would greatly facilitate research, for example high throughput drug screening. Such cells would provide the additional benefit of avoiding the legal and ethical issues presently associated with embryo destruction.

Reprogramming of differentiated cells back into a primitive state is possible by the technique of somatic cell nuclear transfer (SCNT) into an enucleated ovum. See e.g., Hochedlinger et al., Nature 441:1061-7(2006); Wilmut et al., Nature 385:810-3 (1997). More recently, so-called induced pluripotent stem (iPS) cells have been reported to have been generated by recombinant genetic expression of a number of transcription factors (Oct ¾, Sox2, Klf4 and c-Myc or Oct4, Sox2, Nanog, and Lin28) into mouse fibroblasts, generating cells almost indistinguishable from embryonic stem (ES) cells. iPS cells have the morphology of ES cells (formation of colonies), express a gene profile characteristic of ES cells, and are pluripotent in vivo (teratoma formation) and in vitro (embryoid body formation). Similarly, it has been reported that human cells can be induced to pluripotency by these factors, and it appears that the c-Myc gene may not be an essential factor for iPS cell creation. See e.g. Takahashi et al., Cell 131:861-72 (2007); Park et al., Nature 451:141-6 (2008); Lowrey et al., Proc Natl Acad Sci USA 105:2883-8 (2008); Nakagawa et al., Nat Biotechnol 26:101-6 (2008); Wernig et al., Cell Stem Cell 2: 10-2 (2008); and Okita et al., Science 322:949-53 (2008).

Data indicate that autologous iPS cells can be used to treat disease. For example, in one experiment, transcription factor-induced iPS cell reprogramming of autologous tail fibroblasts from sickle cell anemia mice was used in conjunction with gene therapy to correct the genetic defect. The sickle cell mice were subjected to a lethal marrowablating dose of radiation, and rescued by blood cells differentiated from the iPS cells, resulting in cure of the disease. See Hanna et al., Science 318: 1920-3(2007). However, a significant obstacle exists preventing clinical use of iPS cells in that cells transformed using integrating vectors (e.g., retroviruses or lentiviruses) are potentially fatal. For example, in the first human trials of gene therapy, ten boys afflicted by congenital X-linked severe combined immunodeficiency were cured of this condition by replacement of the defective gene, (y chain-c)14, but at least three of the boys subsequently developed leukemia caused by integration of the retrovirus vector in proximity to the proto-oncogene LM02, resulting in its over-expression. See Hacein-Bey-Abina et al., Science 2003; 302:415-9.

Thus, there is exists a need for methods for programming or reprogramming of cells without the use of use of viral vectors. This and other needs are addressed by the compositions, cells, and methods described herein.

### 4. SUMMARY

The methods presented herein generally provide for the generation of a reprogrammed mammalian cell that exhibits at least one characteristic of pluripotency. In certain embodiments, the methods provided herein generate induced pluripotent stem cells. In certain embodiments, the methods comprise the step of contacting a differentiated mammalian cell with one or more transducible polypeptides, wherein a transducible polypeptide comprises the amino acid sequence of a reprogramming factor linked to the amino acid sequence of a cell penetration peptide so that a reprogrammed mammalian cell, e.g., an induced pluripotent stem cell, is generated. In some embodiments, the reprogramming factor is selected from the group consisting of Oct ¾, Sox2, Nanog, Lin28, c-myc and Klf4, e.g., human Oct ¾, human Sox2, human Nanog, human Lin28, human c-myc and human Klf4, and active forms thereof. In some embodiments, at least one reprogramming factor is a human polypeptide. In other embodiments, each of the reprogramming factors is a human polypeptide.

In certain aspects, provided herein is a method for generating a reprogrammed mammalian cell, comprising contacting a differentiated mammalian somatic cell, e.g., a somatic adult cell, with one or more different transducible polypeptides so that a reprogrammed mammalian cell that exhibits at least one characteristic of pluripotency is generated.

In certain aspects, provided herein is a method for generating a reprogrammed mammalian cell, comprising contacting a differentiated mammalian cell in vitro, for example, ex vivo, with one or more different transducible polypeptides so that a reprogrammed mammalian cell that exhibits at least one characteristic of pluripotency is generated.

In certain aspects, provided herein is a method for generating a reprogrammed mammalian cell, comprising contacting a differentiated mammalian somatic cell, e.g., an adult somatic cell, in vitro, for example, ex vivo, with one or more different transducible polypeptides so that a reprogrammed mammalian cell that exhibits at least one characteristic of pluripotency is generated.

In certain embodiments, the transducible polypeptide comprises an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, or a Klf4 polypeptide linked to a cell penetration peptide. In some embodiments, the differentiated mammalian cell is contacted with at least 2, 3, 4, 5 or 6 different transducible polypeptides selected from the group consisting of an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, and a Klf4 polypeptide linked to a cell penetration peptide.

In some embodiments, the cell penetration peptide is selected from the group consisting of herpes simplex virus (HSV) type I protein VP22 (“VP22”), human immunodeficiency virus (HIV-1) transactivator protein TAT (“TAT”), a homeodomain from the Antennapedia polypeptide (“AntP HD”), a polymer of L-arginine or D-arginine amino acid residues (“poly-arginine”), or transducing fragments thereof. In particular embodiments, the cell penetration peptide is VP22. In some embodiments, the cell penetration peptide is linked to the reprogramming factor polypeptide via a peptide bond. In some embodiments, the reprogramming factor polypeptide is linked to the amino terminus, that is, the amino terminal amino acid, of the cell penetration peptide. In other embodiments, the reprogramming factor polypeptide is linked to the carboxy terminus, that is, the carboxy terminal amino acid, of the cell penetration peptide. Linkage can be direct or indirect, e.g., via a linker, such as via a stretch of one or more amino acid residues.

A transducible polypeptide can comprise a single cell penetration peptide or multiple, e.g., 2, 3, 4, 5, or 6 cell penetration peptides. In instances where a transducible polypeptide comprises multiple cell penetration peptides, the cell penetration peptides can be each be of the same sequence, or can vary in sequence.

In certain embodiments, in addition to a reprogramming factor and a cell penetration peptide, a transducible polypeptide can further comprise a purification moiety that can be used in isolation and/or purification of the transducible polypeptide. For example, in certain embodiments, a transducible polypeptide can further comprise a polyhistidine moiety, e.g., six histidine residues, which can, for example, be incorporated at the amino terminal end of the transducible protein. In such an embodiment, the polyhistidine moiety can be used in conjunction with well known nickel-chelate chromatography to isolate and purify the transducible polypeptide.

In other embodiments, in addition to a reprogramming factor and a cell penetration peptide, a transducible polypeptide further comprises a nuclear localization signal (NLS) which enhances nuclear localization of the transducible polypeptide. In some embodiments, the NLS comprises an SV40 large T antigen sequence, e.g., PKKKRKV (SEQ ID NO: 41).

In another aspect, provided herein is a method for generating a reprogrammed mammalian cell, comprising contacting a differentiated mammalian cell with a plurality of different transducible polypeptides so that a reprogrammed mammalian cell that exhibits at least one characteristic of pluripotency is generated. In embodiments where a plurality of different transducible polypeptides is utilized, the cell penetration peptides of the transducible polypeptides can each be of the same sequence or can vary in sequence.

In one embodiment, provided herein is a method for generating a reprogrammed mammalian cell, comprising contacting a differentiated mammalian cell with a plurality of different transducible polypeptides, wherein the different transducible polypeptides comprise: i) an Oct ¾ polypeptide linked to a cell penetration peptide, ii) a Sox2 polypeptide linked to a cell penetration peptide, and iii) a Klf4 polypeptide linked to a cell penetration peptide. In some embodiments, the cell penetration peptide is selected from the group consisting of VP22, TAT, AntP HD, poly-arginine, or transducible fragments thereof. In certain embodiments, the reprogrammed mammalian cell is an induced pluripotent stem cell.

In another embodiment, provided herein is a method for generating a reprogrammed mammalian cell, comprising contacting a differentiated mammalian cell with a plurality of different transducible polypeptides so that a reprogrammed mammalian cell that exhibits at least one characteristic of pluripotency is generated, wherein the different transducible polypeptides comprise: i) an Oct ¾ polypeptide linked to a cell penetration peptide, ii) a Sox2 polypeptide linked to a cell penetration peptide, iii) a c-myc polypeptide linked to a cell penetration peptide, and iv) a Klf4 polypeptide linked to a cell penetration peptide. In some embodiments, the cell penetration peptide is selected from the group consisting of VP22, TAT, AntP HD, poly-arginine, or transducible fragments thereof. In certain embodiments, the reprogrammed mammalian cell is an induced pluripotent stem cell.

In another embodiment, provided herein is a method for generating a reprogrammed mammalian cell, comprising contacting a differentiated mammalian cell with a plurality of different transducible polypeptides so that a reprogrammed mammalian cell that exhibits at least one characteristic of pluripotency is generated, wherein the different transducible polypeptides comprise: i) an Oct ¾ polypeptide linked to a cell penetration peptide, ii) a Sox2 polypeptide linked to a cell penetration peptide, iii) a Nanog polypeptide linked to a cell penetration peptide, and iv) a Lin28 polypeptide linked to a cell penetration peptide. In some embodiments, the cell penetration peptide is selected from the group consisting of VP22, TAT, AntP HD, poly-arginine, or transducing fragments thereof. In certain embodiments, the reprogrammed mammalian cell is an induced pluripotent stem cell.

In another embodiment, provided herein is a method for generating a reprogrammed mammalian cell, comprising contacting a differentiated mammalian cell with a plurality of different transducible polypeptides so that a reprogrammed mammalian cell that exhibits at least one characteristic of pluripotency is generated, wherein the different transducible polypeptides comprise: i) an Oct ¾ polypeptide linked to a cell penetration peptide, ii) a Sox2 polypeptide linked to a cell penetration peptide, iii) a c-myc polypeptide linked to a cell penetration peptide, iv) a Klf4 polypeptide linked to a cell penetration peptide, v) a Nanog polypeptide linked to a cell penetration peptide, and vi) a Lin28 polypeptide linked to a cell penetration peptide. In some embodiments, the cell penetration peptide is selected from the group consisting of VP22, TAT, AntP HD, poly-arginine, or transducible fragments thereof. In some embodiments, the reprogrammed cell is an induced pluripotent stem cell.

In another aspect, provided herein is an isolated reprogrammed mammalian cell that exhibits at least one characteristic of pluripotency, wherein the isolated reprogrammed mammalian cell is generated by a method provided herein.

In another embodiment, provided herein is an isolated reprogrammed mammalian cell that exhibits at least one characteristic of pluripotency, wherein the isolated reprogrammed mammalian cell is generated by a method comprising: contacting a differentiated mammalian cell with one or more different transducible polypeptides so that the reprogrammed mammalian is generated, wherein the one or more different transducible polypeptides comprise an Oct ¾ polypeptide linked to a cell penetration peptide, a Sox2 polypeptide linked to a cell penetration peptide, a Klf4 polypeptide linked to a cell penetration peptide, a c-myc polypeptide linked to a cell penetration peptide, a Nanog polypeptide linked to a cell penetration peptide, or a Lin28 polypeptide linked to a cell penetration peptide. In some embodiments, the isolated reprogrammed mammalian cell is generated by contact with at least 2, 3, 4, 5 or 6 different transducible polypeptides selected from the group consisting of an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, and a Klf4 polypeptide linked to a cell penetration peptide. In some embodiments, the reprogrammed mammalian cell comprises an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, and/or a Klf4 polypeptide linked to a cell penetration peptide. In some embodiments, the reprogrammed mammalian cell does not comprise an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, or a Klf4 polypeptide linked to a cell penetration peptide, but nonetheless exhibits at least one characteristic of pluripotency, e.g., the reprogrammed cell is a cell that has undergone one or more cell divisions, cell doublings or expansions after being generated via the methods presented herein.

In another embodiment, provided herein is an isolated reprogrammed mammalian cell that exhibits at least one characteristic of pluripotency, wherein the isolated reprogrammed mammalian cell is generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, and iii) a Klf4 polypeptide linked to a cell penetration peptide. In some embodiments, the reprogrammed mammalian cell comprises an Oct ¾ polypeptide linked to a cell penetration peptide, a Sox2 polypeptide linked to a cell penetration peptide, and/or a Klf4 polypeptide linked to a cell penetration peptide. In some embodiments, the reprogrammed mammalian cell does not comprise an Oct ¾ polypeptide linked to a cell penetration peptide, a Sox2 polypeptide linked to a cell penetration peptide, or a Klf4 polypeptide linked to a cell penetration peptide, but nonetheless exhibits at least one characteristic of pluripotency, e.g., the reprogrammed cell is a cell that has undergone one or more cell divisions, cell doublings or expansions after being generated via the methods presented herein.

In another embodiment, provided herein is an isolated reprogrammed mammalian cell that exhibits at least one characteristic of pluripotency, wherein the isolated reprogrammed mammalian cell is generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, iii) a c-myc polypeptide linked to a cell penetration peptide, and iv) a Klf4 polypeptide linked to a cell penetration peptide. In some embodiments, the reprogrammed mammalian cell comprises an Oct ¾ polypeptide linked to a cell penetration peptide, a Sox2 polypeptide linked to a cell penetration peptide, a c-myc polypeptide linked to a cell penetration peptide, and/or a Klf4 polypeptide linked to a cell penetration peptide. In some embodiments, the reprogrammed mammalian cell does not comprise an Oct ¾ polypeptide linked to a cell penetration peptide, a Sox2 polypeptide linked to a cell penetration peptide, a c-myc polypeptide linked to a cell penetration peptide, or a Klf4 polypeptide linked to a cell penetration peptide, but nonetheless exhibits at least one characteristic of pluripotency, e.g., the reprogrammed cell is a cell that has undergone one or more cell divisions, cell doublings or expansions after being generated via the methods presented herein.

In another embodiment, provided herein is an isolated reprogrammed mammalian cell that exhibits at least one characteristic of pluripotency, Wherein the isolated reprogrammed mammalian cell is generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, iii) a Nanog polypeptide linked to a cell penetration peptide, and iv) a Lin28 polypeptide linked to a cell penetration peptide. In some embodiments, the reprogrammed mammalian cell comprises an Oct ¾ polypeptide linked to a cell penetration peptide, a Sox2 polypeptide linked to a cell penetration peptide, a Nanog polypeptide linked to a cell penetration peptide, and/or a Lin28 polypeptide linked to a cell penetration peptide. In some embodiments, the reprogrammed mammalian cell does not comprise an Oct ¾ polypeptide linked to a cell penetration peptide, a S6x2 polypeptide linked to a cell penetration peptide, a Nanog polypeptide linked to a cell penetration peptide, or a Lin28 polypeptide linked to a cell penetration peptide, but nonetheless exhibits at least one characteristic of pluripotency, e.g., the reprogrammed cell is a cell that has undergone one or more cell divisions, cell doublings or expansions after being generated via the methods presented herein.

In another embodiment, provided herein is an isolated reprogrammed mammalian cell that exhibits at least one characteristic of pluripotency, wherein the isolated reprogrammed mammalian cell is generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, iii) a c-myc polypeptide linked to a cell penetration peptide, iv) a Klf4 polypeptide linked to a cell penetration peptide, v) a Nanog polypeptide linked to a cell penetration peptide, and vi) a Lin28 polypeptide linked to a cell penetration peptide. In some embodiments, the reprogrammed mammalian cell comprises an Oct ¾ polypeptide linked to a cell penetration peptide, a Sox2 polypeptide linked to a cell penetration peptide, a c-myc polypeptide linked to a cell penetration peptide, a Klf4 polypeptide linked to a cell penetration peptide, a Nanog polypeptide linked to a cell penetration peptide, and/or a Lin28 polypeptide linked to a cell penetration peptide. In some embodiments, the reprogrammed mammalian cell does not comprise an Oct ¾ polypeptide linked to a cell penetration peptide, a Sox2 polypeptide linked to a cell penetration peptide, a c-myc polypeptide linked to a cell penetration peptide, a Klf4 polypeptide linked to a cell penetration peptide, a Nanog polypeptide linked to a cell penetration peptide, or a Lin28 polypeptide linked to a cell penetration peptide, but nonetheless exhibits at least one characteristic of pluripotency, e.g., the reprogrammed cell is a cell that has undergone one or more cell divisions, cell doublings or expansions after being generated via the methods presented herein.

In another aspect, provided herein is an isolated population of cells comprising at least 70%, at least 80%, at least 90%, or at least 95% reprogrammed mammalian cells that exhibit at least one characteristic of pluripotency , wherein the reprogrammed mammalian cells are generated by a method provided herein. In one embodiment, the isolated population of cells comprises about 1×10⁵, 5×10⁵, 1×10⁶, 5×10⁶, 1×10⁷, 5×10⁷, 1×10⁸, or 4×10⁸ reprogrammed cells or total cells. In one embodiment, the isolated population of cells is present in a formulation suitable for administration to a human. In another embodiment, the isolated population of cells is present in a bag, e.g., a plastic bag, such as a plastic bag suitable for use in administration of the cells to a human. In another embodiment, the isolated population of cells is present in a syringe, such as a sterile syringe suitable for administration of the cells to a human.

In another embodiment, provided herein is an isolated population of cells comprising at least 70%, at least 80%, at least 90%, or at least 95% reprogrammed mammalian cells that exhibit at least one characteristic of pluripotency, wherein the reprogrammed mammalian cells are generated by a method comprising: contacting a differentiated mammalian cell with one or more different transducible polypeptides so that the reprogrammed mammalian cell is generated, wherein the one or more different transducible polypeptides comprise an Oct ¾ polypeptide linked to a cell penetration peptide, a Sox2 polypeptide linked to a cell penetration peptide, a Klf4 polypeptide linked to a cell penetration peptide, a c-myc polypeptide linked to a cell penetration peptide, a Nanog polypeptide linked to a cell penetration peptide, or a Lin28 polypeptide linked to a cell penetration peptide. In some embodiments, the reprogrammed mammalian cells are generated by contact with at least 2, 3, 4, 5 or 6 different transducible polypeptides selected from the group consisting of an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, or a Klf4 polypeptide linked to a cell penetration peptide. Also provided herein are such isolated populations of cells further comprising at least one other isolated population of cells, e.g., stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In some embodiments, the reprogrammed mammalian cells of such isolated populations comprise an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, and/or a Klf4 polypeptide linked to a cell penetration peptide. In some embodiments, at least a portion of the reprogrammed mammalian cells do not comprise an Oct ¾ polypeptide linked to a cell penetration peptide, a Sox2 polypeptide linked to a cell penetration peptide, a Nanog polypeptide linked to a cell penetration peptide, a Lin28 polypeptide linked to a cell penetration peptide, a c-myc polypeptide linked to a cell penetration peptide, or a Klf4 polypeptide linked to a cell penetration peptide, but nonetheless exhibit at least one characteristic of pluripotency, e.g., the reprogrammed cell is a cell that has undergone one or more cell divisions, cell doublings or expansions after being generated via the methods presented herein.

In another embodiment, provided herein is an isolated population of cells comprising at least 70%, at least 80%, at least 90%, or at least 95% reprogrammed mammalian cells that exhibit at least one characteristic of pluripotency, wherein the reprogrammed mammalian cells are generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, and iii) a Klf4 polypeptide linked to a cell penetration peptide. Also provided herein are such isolated populations of cells further comprising at least one other isolated population of cells, e.g., stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In some embodiments, the reprogrammed mammalian cells of such populations of cells comprise an Oct ¾ polypeptide linked to a cell penetration peptide, a Sox2 polypeptide linked to a cell penetration peptide, and/or a Klf4 polypeptide linked to a cell penetration peptide. In some embodiments, at least a portion of the reprogrammed mammalian cells do not comprise an Oct ¾ polypeptide linked to a cell penetration peptide, a Sox2 polypeptide linked to a cell penetration peptide, or a Klf4 polypeptide linked to a cell penetration peptide, but nonetheless exhibit at least one characteristic of pluripotency, e.g., the reprogrammed cell is a cell that has undergone one or more cell divisions, cell doublings or expansions after being generated via the methods presented herein.

In another embodiment, provided herein is a isolated population of cells comprising at least 70%, at least 80%, at least 90%, or at least 95% reprogrammed mammalian cells that exhibit at least one characteristic of pluripotency, wherein the reprogrammed mammalian cells are generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, iii) a c-myc polypeptide linked to a cell penetration peptide, and iv) a Klf4 polypeptide linked to a cell penetration peptide. Also provided herein are such isolated populations of cells further comprising at least one other isolated population of cells, e.g., stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In some embodiments, the reprogrammed mammalian cells of such populations of cells comprise an Oct ¾ polypeptide linked to a cell penetration peptide, a Sox2 polypeptide linked to a cell penetration peptide, a c-myc polypeptide linked to a cell penetration peptide, and/or a Klf4 polypeptide linked to a cell penetration peptide. In some embodiments, at least a portion of the reprogrammed mammalian cells do not comprise an Oct ¾ polypeptide linked to a cell penetration peptide, a Sox2 polypeptide linked to a cell penetration peptide, a c-myc polypeptide linked to a cell penetration peptide, or a Klf4 polypeptide linked to a cell penetration peptide, but nonetheless exhibit at least one characteristic of pluripotency, e.g., the reprogrammed cell is a cell that has undergone one or more cell divisions, cell doublings or expansions after being generated via the methods presented herein.

In another embodiment, provided herein is a isolated population of cells comprising at least 70%, at least 80%, at least 90%, or at least 95% reprogrammed mammalian cells that exhibit at least one characteristic of pluripotency, wherein the reprogrammed mammalian cells are generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, iii) a Nanog polypeptide linked to a cell penetration peptide, and iv) a Lin28 polypeptide linked to a cell penetration peptide. Also provided herein are such isolated ed populations of cells further comprising at least one other isolated population of cells, e.g., stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In some embodiments, the reprogrammed mammalian cells of such populations of cells comprise an Oct ¾ polypeptide linked to a cell penetration peptide, a Sox2 polypeptide linked to a cell penetration peptide, a Nanog polypeptide linked to a cell penetration peptide, and/or a Lin28 polypeptide linked to a cell penetration peptide. In some embodiments, at least a portion of the reprogrammed mammalian cells do not comprise an Oct ¾ polypeptide linked to a cell penetration peptide, a Sox2 polypeptide linked to a cell penetration peptide, a Nanog polypeptide linked to a cell penetration peptide, or a Lin28 polypeptide linked to a cell penetration peptide, but nonetheless exhibit at least one characteristic of pluripotency, e.g., the reprogrammed cell is a cell that has undergone one or more cell divisions, cell doublings or expansions after being generated via the methods presented herein.

In another embodiment, provided herein is an isolated population of cells comprising at least 70%, at least 80%, at least 90%, or at least 95% reprogrammed mammalian cells that exhibit at least one characteristic of pluripotency, wherein the reprogrammed mammalian cells are generated by a method comprising: contacting a differentiated mammalian cell with a plurality of different transducible polypeptides so that the reprogrammed mammalian cell is generated, wherein the different transducible polypeptides comprise: i) an Oct ¾ polypeptide linked to a cell penetration peptide, ii) a Sox2 polypeptide linked to a cell penetration peptide, iii) a c-myc polypeptide linked to a cell penetration peptide, iv) a Klf4 polypeptide linked to a cell penetration peptide, v) a Nanog polypeptide linked to a cell penetration peptide, and vi) a Lin28 polypeptide linked to a cell penetration peptide. In some embodiments, the reprogrammed mammalian cells comprise an Oct ¾ polypeptide linked to a cell penetration peptide, a Sox2 polypeptide linked to a cell penetration peptide, a c-myc polypeptide linked to a cell penetration peptide, a Klf4 polypeptide linked to a cell penetration peptide, a Nanog polypeptide linked to a cell penetration peptide, and/or a Lin28 polypeptide linked to a cell penetration peptide.

In some embodiments, the reprogrammed mammalian cells of such populations of cells do not comprise an Oct ¾ polypeptide linked to a cell penetration peptide, a Sox2 polypeptide linked to a cell penetration peptide, a c-myc polypeptide linked to a cell penetration peptide, a Klf4 polypeptide linked to a cell penetration peptide, a Nanog polypeptide linked to a cell penetration peptide, or a Lin28 polypeptide linked to a cell penetration peptide, but nonetheless exhibit at least one characteristic of pluripotency, e.g., the reprogrammed cell is a cell that has undergone one or more cell divisions, cell doublings or expansions after being generated via the methods presented herein. Also provided herein are such isolated populations of cells further comprising at least one other isolated population of cells, e.g., stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In another aspect, the invention provides a method for cardiac differentiation of a mammalian cell comprising: contacting a mammalian cell which exhibits at least one characteristic of pluripotency with a transducible polypeptide so that cardiac differentiation of the mammalian cell occurs. In one embodiment, the transducible polypeptide comprises an islet 1 (ISL 1) polypeptide, e.g., a human ISL 1 polypeptide, linked to a cell penetration peptide. In some embodiments, the mammalian cell is a pluripotent cell, e.g., an induced pluripotent stem cell, an embryonic stem cell, or an adult stem cell, such as a cardiac stem cell, e.g., a cardiosphere-derived stem cell. In certain embodiments, the cell is a reprogrammed cell generated via methods provided herein. In some embodiments, the mammalian cell is a human cell.

In several embodiments, a method for generating a reprogrammed mammalian cell without the use of a virus is provided. Viruses include, but are not limited to, retroviruses, lentiviruses, adenoviruses, and adeno-associated viruses. In one embodiment, the method comprises contacting a differentiated mammalian cell with transducible polypeptides. The transducible polypeptides comprise an Oct ¾ polypeptide linked to a cell penetration peptide, a Sox2 polypeptide linked to a cell penetration peptide, a NANOG polypeptide linked to a cell penetration peptide, and a Lin28 polypeptide linked to a cell penetration peptide. In another embodiment, the transducible polypeptides comprise an Oct ¾ polypeptide linked to a cell penetration peptide, a Sox2 polypeptide linked to a cell penetration peptide, and a Klf4 polypeptide linked to a cell penetration peptide. Optionally, a c-myc polypeptide linked to a cell penetration peptide is further provided in some embodiments. In one embodiment, the cell penetration peptide comprises an amino terminus and a carboxy terminus. In several embodiments, the method generates reprogrammed cells that exhibit at least one characteristic of pluripotency.

In several embodiments, a virus-free composition for generating a reprogrammed mammalian cell exhibiting at least one characteristic of pluripotency is provided. Virus-free compositions include, but are not limited to, compositions that do not contain significant amounts of (i) replication-competent viruses; and/or (ii) viruses that randomly integrate into the host genome; and/or (iii) whole viruses, and/or (iv) pathogenic viruses; and/or (v) immunogenic viruses. Virus-free compositions may include viral particles that (i) are not replication-competent; and/or (ii) do not randomly integrate into the host genome; and/or (iii) are not pathogenic; and/or (iv) are not immunogenic. In one embodiment, the composition comprises transducible polypeptides for contacting a differentiated mammalian cell. The transducible polypeptides comprise an Oct ¾ polypeptide linked to a cell penetration peptide, a Sox2 polypeptide linked to a cell penetration peptide, a NANOG polypeptide linked to a cell penetration peptide, and a Lin28 polypeptide linked to a cell penetration peptide. In another embodiment, the transducible polypeptides comprise an Oct ¾ polypeptide linked to a cell penetration peptide, a Sox2 polypeptide linked to a cell penetration peptide, and a Klf4 polypeptide linked to a cell penetration peptide. Optionally, a c-myc polypeptide linked to a cell penetration peptide is further provided in some embodiments. In one embodiment, the cell penetration peptide comprises an amino terminus and a carboxy terminus. In some embodiments, the polypeptides are joined and/or synthesized (e.g., in frame) with one another (or are linked with optional linker molecules), and include one or more cell penetration peptides.

In another embodiment, the invention provides a method for cardiac differentiation of a mammalian cell comprising: contacting a mammalian cell which exhibits at least one characteristic of pluripotency in vitro, for example ex vivo, with a transducible polypeptide so that cardiac differentiation of the mammalian cell occurs. In one embodiment, the transducible polypeptide comprises an islet 1 (ISL 1) polypeptide, e.g., a human ISL 1 polypeptide, linked to a cell penetration peptide. In some embodiments, the mammalian cell is a pluripotent cell, e.g., an induced pluripotent stem cell, an embryonic stem cell, or an adult stem cell, such as a cardiac stem cell, e.g., a cardiosphere-derived stem cell. In certain embodiments, the cell is a reprogrammed cell generated via methods provided herein. In some embodiments, the mammalian cell is a human cell.

### 5. BRIEF DESCRIPTION OF THE FIGURES

FIG. 1 presents quantitative RT-PCR data demonstrating marked upregulation of collagen transcription (COL1A) in cardiac tissue following LAD ligation surgery. The data confirms myocardial infarction of the cardiac tissue.

FIG. 2A presents Western blot data of ISL 1 protein. Lanes 1 and 2 are control mouse hearts, and lanes 3 and 4 are fourteen day post-myocardial infarction hearts.

FIG. 2B presents densitometry data of ISL-1 Western blot signal from five control and five 14-day post-infarction hearts. p<0.01.

FIG. 2C presents quantitative RT-PCR data which demonstrates upregulation of ISL 1 transcription following myocardial infarction. Solid line indicates post-myocardial infarction hearts, dotted line indicates control hearts (n=5 each group).

FIG. 2D presents Western blotting which demonstrates upregulation of c-kit following myocardial infarction. Top panel indicates densitometry of Western blot bands (n=5 in each group, p<0.05). Bottom panel shows representative bands.

FIG. 3 presents immunofluorescence data of peri-infarct tissue. The cells that produce Isl1 within the peri-infarct tissue are c-kit positive cells. Red labels c-kit, green indicates Isl1-1 and blue labels the nucleus (DAPI).

FIG. 4A presents PCR amplification of the ISL 1 gene. The mouse ISL 1 gene was amplified by PCR (Taq polymerase, Invitrogen), using plasmid DNA as the template (Open Biosystems cat #EMM1O02-99258597).

FIG. 4B presents sequencing chromatograms for the region spanning the junction between the VP22-coding region and the ISL 1-coding region of a polynucleotide encoding ISL1-VP22. The PCR product of FIG. 4A was ligated into the expression plasmid by the topoisomerase reaction (Voyager protein expression kit, Invitrogen) The resultant plasmid was sequenced to confirm that the PCR product had inserted in frame with the VP22 gene, and in the correct orientation.

FIG. 4C presents the expression of the ISL1-VP22 hybrid protein by induction of E coli by isopropyl 13-D-thiogalactoside. The resulting protein was then purified using ProBond elution columns (Invitrogen).

FIG. 4D presents Western blot data of VP22 and ISL1-VP22 purified proteins. The synthesized VP22 and ISL1-VP22 proteins were confirmed to be the correct size (23 and 62 kDa respectively).

FIG. 5 presents the percentage of beating embryoid bodies in proportion of beating embryoid bodies was markedly increased by exposure of the cells to ISL1-VP22 hybrid protein, compared to exposure to VP22 protein alone, or no additional protein.

### 6. DETAILED DESCRIPTION OF THE EMBODIMENTS

### 6.1 Terminology

As used herein, a cell exhibits “at least one characteristic of pluripotency” if the cell: expresses at least one marker, as detected by RT-PCR, cell sorting, or immunocytochemistry techniques, of pluripotency selected from the group consisting of SSEA-3, SSEA-4, TRA-1-60, Nanog, and Oct ¾; displays the ability to form embryoid bodies in vitro; can form tightly packed colonies on culture plates; displays the ability to differentiate into cells having characteristics of endoderm, mesoderm or ectoderm when injected into SCID mice; displays the ability to form teratomas if injected into animals; exhibits an exponential pattern of growth in cell culture, without senescence; exhibits telomerase activity; exhibits the ability to undergo at least between 10-40 population doublings in culture; comprises unmethylated DNA characteristic of pluripotent clones; or displays germline competence. It is noted that a cell that exhibits at least one characteristic of pluripotency can include, for example, a multipotent cell or a pluripotent cell. It is further noted, in the context of reprogrammed cells, that in instances whereby the differentiated mammalian cell used in generating the reprogrammed cell itself exhibits at least one characteristic of pluripotency, the resulting reprogrammed mammalian cell exhibits at least one additional characteristic of pluripotency relative to the differentiated mammalian cell, and/or exhibits quantitatively more of at least one characteristic of pluripotency relative to the differentiated mammalian cell.

As used herein, the term “pluripotent cell” refers to a cell that has complete differentiation versatility, i.e., the capacity to grow into any of the mammalian body's approximately 260 cell types.

As used herein, the term “multipotent cell” refers to a cell that has the capacity to grow into any of subset of the mammalian body's approximately 260 cell types. Certain multi potent cells can differentiate into at least one cell type of ectoderm, mesoderm, and endoderm germ layers.

As used herein, the term “differentiated cell,” in the context of mammalian cells, refers to any cell undergoing or having undergone differentiation into a somatic cell lineage. The term encompasses both partially differentiated and terminally differentiated cells. A partially differentiated cell is not a pluripotent cell. A terminally differentiated cell generally does not exhibit at least one characteristic of pluripotency.

As used herein the term “cell penetration peptide” refers to an amino acid sequence that, when linked to a polypeptide, e.g., a reprogramming factor, causes or enhances the ability of the polypeptide to cross the cell membrane of a cell when the cell is contacted by the cell penetration peptide linked to the polypeptide. A “transducing fragment” of a cell penetration peptide, refers to a portion of a full-length cell penetration peptide, e.g., a portion of a VP22, TAT, or ANTP HD sequence, that, when linked to a polypeptide, e.g., a reprogramming factor, causes or enhances the ability of the polypeptide to cross the cell membrane of a cell when the cell is contacted by the transducing fragment linked to the polypeptide.

As used herein, a “reprogramming factor” refers to a factor, e.g., a polypeptide, that when introduced to a differentiated mammalian cell causes, induces, enhances, or contributes to generation of a reprogrammed cell from the contacted differentiated cell.

### 6.2 Methods For Generating a Reprogrammed Mammalian Cell

6.2.1 Reprogramming Factors

In some embodiments, the transducible polypeptide useful for the methods provided herein comprises a cell penetration peptide linked with an Oct ¾ polypeptide, e.g., a human or mouse Oct ¾ polypeptide. In particular embodiments, the cell penetration peptide is selected from the group comprising VP22, TAT, Antp HD, and poly-arginine. The sequences of human and mouse Oct ¾ have been described previously. See, e.g., Yeom et al., Mech. Dev. 35 (3), 171-179 (1991); Takeda et al., Nucleic Acids Res. 20 (17), 4613-4620 (1992). Representative cDNA sequences of human Oct ¾ are provided herein as SEQ ID NOS: 1 and 2, and representative amino acid sequences of human Oct ¾ are provided as SEQ 10 NOS: 3 and 4. In a particular embodiment, the transducible polypeptide comprises the carboxy terminus of VP22 linked to the amino terminus of Oct ¾, such as the transducible polypeptide provided herein as SEQ 10 NO:25. In another particular embodiment, the transducible polypeptide comprises the carboxy terminus of Oct ¾ linked to the amino terminus of VP22, such as the transducible polypeptide provided herein as SEQ ID NO:26. The linkage can be direct or indirect.

In some embodiments, the transducible polypeptide useful for the methods provided herein comprises a cell penetration peptide fused with a Sox2 polypeptide, e.g., a human or mouse Sox2 polypeptide. In particular embodiments, the cell penetration peptide is selected from the group comprising VP22, TAT, Antp HD, and poly-arginine. The sequences of human and mouse Sox2 have been described previously. See, e.g., Gubbay et al., Nature, 6281:245-50 (1990); Stevanovic et al., Mamm. Genome 5 (10), 640-642 (1994). Representative cDNA and amino acid sequences of human Sox2 are provided herein as SEQ ID NOS: 5 and 6, respectively. In a particular embodiment, the transducible polypeptide comprises the carboxy terminus of VP22 linked to the amino terminus of Sox2, such as the transducible polypeptide provided herein as SEQ ID NO:27. In another particular embodiment, the transducible polypeptide comprises the carboxy terminus of Sox2 linked to the amino terminus of VP22, such as the transducible polypeptide provided herein as SEQ 10 NO:28. The linkage can be direct or indirect.

In some embodiments, the transducible polypeptide useful for the methods provided herein comprises a cell penetration peptide linked with a Nanog polypeptide, e.g., a human or mouse Nanog polypeptide. Nanog is a homeodomain-bearing transcription factor. In particular embodiments, the cell penetration peptide is selected from the group comprising VP22, TAT, Antp HD, and poly-arginine. The sequences of human and mouse Nanog have been described previously. See, e.g., Mitsui et al., Cell. 2003 May 30; 113(5):631-42; Chambers et al., Cell. 2003 May 30; 113(5):643-55. Representative cDNA and amino acid sequences of human Nanog are provided herein as SEQ 10 NOS: 7 and 8, respectively. In a particular embodiment, the transducible polypeptide comprises the carboxy terminus of VP22 linked to the amino terminus of Nanog, such as the transducible polypeptide provided herein as SEQ 10 NO:29. In another particular embodiment, the transducible polypeptide comprises the carboxy terminus of Nanog linked to the amino terminus of VP22, such as the transducible polypeptide provided herein as SEQ 10 NO:30. The linkage can be direct or indirect.

In some embodiments, the transducible polypeptide useful for the methods provided herein comprises a cell penetration peptide linked with a Lin28 polypeptide, e.g., a human or mouse Lin28 polypeptide. In particular embodiments, the cell penetration peptide is selected from the group comprising VP22, TAT, Antp HD, and poly-arginine. The sequences of human and mouse Lin28 have been described previously. See, e.g., Moss et al., Dev. Biol. 258 (2), 432-442 (2003); Sempere et al., Genome Biol. 5 (3), R13 (2004). Representative cDNA and amino acid sequences of human Lin28 are provided herein as SEQ ID NOS: 9 and 10, respectively. In a particular embodiment, the transducible polypeptide comprises the carboxy terminus of VP22 linked to the amino terminus of Lin28, such as the transducible polypeptide provided herein as SEQ ID NO:31. In another particular embodiment, the transducible polypeptide comprises the carboxy terminus of Lin28 linked to the amino terminus of VP22, such as the transducible polypeptide provided herein as SEQ ID NO:32. The linkage can be direct or indirect.

In some embodiments, the transducible polypeptide useful for the methods provided herein comprises a cell penetration peptide linked with a c-myc polypeptide, e.g., a human or mouse c-myc polypeptide. In particular embodiments, the cell penetration peptide is selected from the group comprising VP22, TAT, Antp HD, and poly-arginine. The sequences of human and mouse c-myc have been described previously. See, e.g., Himing-Folz et al., Cytogenet. Cell Genet. 61 (4), 289-294 (1992); Takahashi et al., Cytogenet. Cell Genet. 57 (2-3), 109-111 (1991). Representative cDNA and amino acid sequences of human c-myc are provided herein as SEQ ID NOS: II and 12, respectively. In a particular embodiment, the transducible polypeptide comprises the carboxy terminus of VP22 linked to the amino terminus of c-myc, such as the transducible polypeptide provided herein as SEQ ID NO:33. In another particular embodiment, the transducible polypeptide comprises the carboxy terminus of c-myc linked to the amino terminus of VP22, such as the transducible polypeptide provided herein as SEQ ID NO:34. The linkage can be direct or indirect.

In some embodiments, the transducible polypeptide useful for the methods provided herein comprises a cell penetration peptide linked with a Klf4 polypeptide, e.g., a human or mouse Klf4 polypeptide. In particular embodiments, the cell penetration peptide is selected from the group comprising VP22, TAT, Antp HD, and poly-arginine. The sequences of human and mouse Klf4 have been described previously. See e.g., Shields et al., J Biol. Chem. 271 (33), 20009-20017 (1996); Conkright et al., Nucleic Acids Res. 27 (5), 1263-1270 (1999). Representative cDNA and amino acid sequences of human Klf4 are provided herein as SEQ ID NOS: 13 and 14, respectively. In a particular embodiment, the transducible polypeptide comprises the carboxy terminus of VP22 linked to the amino terminus of K1f4, such as the transducible polypeptide provided herein as SEQ ID NO:35. In another particular embodiment, the transducible polypeptide comprises the carboxy terminus of Klf4 linked to the amino terminus of VP22, such as the transducible polypeptide provided herein as SEQ ID NO:36. The linkage can be direct or indirect.

6.2.2 Cell Penetration Peptides

Among the cell penetration peptides useful for the methods provided herein is the Herpes simplex type I virus (HSV1) virion protein VP22. See, e.g., Elliot & O'Hare, 88 Cell 223-233 (1997) and PCT International patent application WO 97/97/05265). A representative full length VP22 sequence (aa 1-301) is depicted herein (SEQ ID NO: 15).

Sequences of VP22 that can be utilized as transducing fragments of VP22 are also well known. See, e.g., PCT International patent applications WO 97/05265, WO 98/04708, and WO 98/32866, each of which is incorporated herein by reference. Such sequences can include, for example, amino acid sequences corresponding to amino acids 60-301 and 159-301 of the full-length HSVI VP22 sequence (SEQ ID NO: 16).

VP22 sequences that can be used in conjunction with the methods described herein extend to homologous proteins and transducing fragments thereof based on sequences of VP22 protein homo logs from other herpesviruses. For example, VP22-homolog sequences have been obtained from VZV (e.g., all or homologous parts of the sequence from aa 1-302), from MDV (e.g., all or homologous parts of the sequence from aa 1-249), and from BHV (e.g., all or homologous parts of the sequence from aa 1-258) (see PCT International Publication Nos. WO 97/05265, WO 98/04708, and WO 98/32866). The sequences of the corresponding proteins from HSV2, VZV, BHV and MDV are well known and available in public protein/nucleic acid sequence databases. Thus, for example, within the EMBL/Genbank database, a VP22 sequence from HSV2 is available as gene item UL49 under accession no. Z86099 containing the complete genome of HSV2 strain HG52; the complete genome of VZV including the homologous gene/protein is available under accession numbers X04370, M14891, M16612; the corresponding protein sequence from BHV is available as “bovine herpesvirus 1 virion tegument protein” under accession number U21137; and the corresponding sequence from MDV is available as gene item UL49 under accession number LI 0283 for “gallid herpesvirus type I homologous sequence genes.” In these proteins, especially those from HSV2 and VZV, corresponding deletions can be made, e.g. of sequences homologous to aa 1-60 or aa 1-159 of VP22 from HSV 1. These cited sequences are hereby incorporated herein by reference.

Transducing fragments of VP22 can also, for example, contain one or a plurality of amino acid sequence motifs or their homologs from the C-terminal sequence of VP22 of HS I or other herpesviruses, which can be selected from RSASR (SEQ [0 NO: 17), RTASR (SEQ 10 NO:18), RSRAR (SEQ 10 NO:19), RTRAR (SEQ 10 NO:20), ATATR (SEQ 10 NO:21), and wherein the third or fourth residue A can be duplicated, e.g., as in RSAASR (SEQ 10 NO:22).

Among the cell penetration peptides useful for the methods provided herein is the human immunodeficiency virus (HIV-1) TAT protein. The sequences of HIV-I TAT polypeptides are well known. See, e.g., (Frankel & Pabo, Cell 55:1189-93 (1988); Green & Loewenstein, Cell 55:1179-88 (1988)).

Sequences of HIV-I TAT that can be used as transducing fragments are also well known. For example, in certain embodiments, a cell penetration peptide the YGRKKRRQRRR (SEQ 10 NO:23) HIV-1 TAT amino acid sequence.

Among the cell penetration peptides useful for the methods provided herein is the homeodomain of the Drosophila melanogaster protein Antennapedia (Antp HD) (Lindsay, Curr. Op. Pharmacol. 2:587-94 (2002); Derossi et al., J. Biol. Chem. 269:10444-50 (1994)), described, e.g., in PCT Publication Nos. WO 97/12912 and WO 99/11809.

Sequences of Antp HD that can be utilized as transducing fragments are also well known. For example, among such sequences is RQIKIWFQNRRMKWKK (SEQ ID NO: 24), corresponding to the third helix of the Antp HD homeodomain.

Among the cell penetration peptides useful for the methods provided herein are sequences containing arginine (Arg) repeats, or poly-arginine. For example, such cell penetration peptides can comprise contiguous or partially contiguous segments of at least 5, 6, 7, 8, 9, 10, 11, 12, 15, 20, 25, 50, 100, or 1000 arginine residues, wherein the arginine residues may be of the D-form, the L-form, or mixtures of each.

6.2.3 Methods of Making Transducible Polypeptides

As discussed herein, a transducible polypeptide comprises a reprogramming factor linked to a cell penetration peptide, and can optionally comprise a nuclear localization signal and/or a purification moiety. Transducible polypeptides provided herein can be made using any of a variety of methods, e.g., recombinant or synthetic methods, well known to those of skill in the art.

In one aspect, a transducible polypeptide can be made using standard recombinant DNA techniques. For example, a polynucleotide comprising the coding sequence of a transducible polypeptide, e.g., the coding sequence for a cell penetration peptide joined in-frame with the coding sequence of a reprogramming factor in an expression vector, e.g., a plasmid vector, and can be expressed in any suitable cell, e.g., a bacterial or mammalian cell. Techniques are also well known for isolating and purifying polypeptides expressed via such methods from the expressing cells and from the media used during culture of the expressing cells.

In certain embodiments, a cell is used to express a single transducible polypeptide. In other embodiments, a cell is engineered to express greater than one form of transducible polypeptide, e.g., is engineered to express a transducible polypeptide comprising a Oct ¾ reprogramming factor, and also a transducible polypeptide comprising a Sox2 reprogramming factor.

In certain embodiments, in addition to the coding sequence of a reprogramming factor and one or more cell penetration peptides, the coding sequence further comprises the amino acid sequence of a purification moiety. The location of the coding sequence of the purification moiety can be placed in any position that does not interfere with the expression or activity of the reprogramming factor, the cell penetration peptide, or the optional nuclear localization sequence if it is to be present on the transducible polypeptide. For example, the coding sequence can be placed upstream (5′) or downstream (3′) of the coding sequence of the coding sequence of the reprogramming factor such that the purification moiety is amino or carboxy to the reprogramming factor in the expressed transducible polypeptide, respectively. Likewise, the coding sequence can be placed upstream (5′) or downstream (3′) of the coding sequence of the coding sequence of the cell penetration peptide such that the purification moiety is amino or carboxy to the cell penetration peptide in the expressed transducible polypeptide, respectively. In particular embodiments, the purification moiety is present at the amino terminal end of the transducible polypeptide.

In certain embodiments, in addition to the coding sequence of a reprogramming factor and one or more cell penetration peptides, the coding sequence further comprises the amino acid sequence of a nuclear localization sequence (NLS). The location of the coding sequence of the NLS can be placed in any position that does not interfere with the expression or activity of the reprogramming factor, the cell penetration peptide, or the optional purification moiety, if it is to be present on the transducible polypeptide. For example, the coding sequence can be placed upstream (5′) or downstream (3′) of the coding sequence of the coding sequence of the reprogramming factor such that the NLS is amino or carboxy to the reprogramming factor in the expressed transducible polypeptide, respectively. Likewise, the coding sequence can be placed upstream (5′) or downstream (3′) of the coding sequence of the cell penetration peptide such that the NLS is amino or carboxy to the cell penetration peptide in the expressed transducible polypeptide, respectively.

In certain embodiments, the transducible polypeptide comprises a reprogramming factor linked to the amino terminus of a cell penetration peptide. In such embodiments, the coding sequence of the transducible polypeptide is arranged accordingly. Thus, in one embodiment, the coding sequence of the reprogramming factor is positioned in-frame with the coding sequence of the cell penetration peptide such that the carboxy-most amino acid residue of the reprogramming factor is adjacent to the amino-most amino acid residue of the cell penetration peptide in the expressed transducible polypeptide. In an alternate embodiment, the coding sequence of the reprogramming factor is positioned in-frame with the coding sequence of an amino acid linker sequence, which is, in turn, positioned in-frame with the coding sequence of the cell penetration peptide. In such an embodiment, the amino acid sequence of the reprogramming factor is also linked to the amino terminus of the cell penetration peptide, but is linked via the linker sequence.

Likewise, in certain embodiments, the transducible polypeptide comprises a reprogramming factor linked to the carboxy terminus of a cell penetration peptide. In such embodiments, the coding sequence of the transducible polypeptide is arranged accordingly. Thus, in one embodiment, the coding sequence of the cell penetration peptide is positioned in-frame with the coding sequence of the reprogramming factor such that the carboxy-most amino acid residue of the cell penetration peptide is adjacent to the amino-most amino acid residue of the reprogramming factor in the expressed transducible polypeptide. In an alternate embodiment, the coding sequence of the cell penetration peptide is positioned in-frame with the coding sequence of an amino acid linker sequence, which is, in turn, positioned in-frame with the coding sequence of the reprogramming factor. In such an embodiment, the amino acid sequence of the reprogramming factor is also linked to the carboxy terminus of the cell penetration peptide, but is linked via the linker sequence.

Techniques for construction of expression vectors and expression of genes in cells comprising the expression vectors are well known in the art. See, e.g., Sambrook et al., 200 I, Molecular Cloning—A Laboratory Manual, 3rd edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

Useful promoters for use in expression vectors include, but are not limited to, a metallothionein promoter, a constitutive adenovirus major late promoter, a dexamethasone-inducible MMTV promoter, a SV40 promoter, a MRP pol III promoter, a constitutive MPSV promoter, a tetracycline-inducible CMV promoter (such as the human immediate-early CMV promoter), and a constitutive CMV promoter.

The expression vectors should contain expression and replication signals compatible with the cell in which the transducible polypeptides are expressed. Expression vectors useful for transducible constructs include viral vectors such as retroviruses, adenoviruses and adenoassociated viruses, plasmid vectors, cosmids, and the like. Viral and plasmid vectors are preferred for transfecting the expression vectors into mammalian cells. For example, the expression vector pcDNAI (Invitrogen, San Diego, Calif.), in which the expression control sequence comprises the CMV promoter, provides good rates of transfection and expression into such cells.

Transducible polypeptides can also be made, for example, using chemical synthetic methods, or a combination of synthetic and recombinant methods. For example, transducible polypeptides can be synthetically produced using standard polypeptide synthesis techniques well known by those of skill in the art. Alternatively, portions of a transducible polypeptide can be purified, or recombinantly expressed using, e.g., techniques such as those described herein, and the portions can be linked using synthetic techniques to yield complete transducible polypeptides.

In embodiments in which portions of a transducible polypeptide are expressed or purified and then linked, the linkage can be via covalent, e.g., peptide bond, or noncovalent linkage, and can be direct or via a linker moiety, e.g., a linker moiety that links a reprogramming factor with a cell penetration peptide.

Any of a variety of linkages can be utilized, including, but not limited to ether, ester, thioether, thioester, amide, imide, disulfide, peptide, or other linkages. Linkage can be likewise be via any of a variety of functional groups, for example, sulfhydryl (—S), carboxylic acid (COOH) or free amine (—NH2) groups. The skilled artisan can routinely select the appropriate linkage, optional linker, and method for attaching the linking the portions of the transducible polypeptide based, for example, on the physical and chemical properties of the elements, e.g., the cell penetration peptide and/or the reprogramming factor, of the transducible polypeptide.

In embodiments where a linker is utilized, the linker can directly link portions of the transducible polypeptide, e.g., a cell penetration peptide and a reprogramming factor polypeptide. In other embodiments, the linker itself can comprises two or more molecules that associate to link portions of the transducible polypeptide, e.g., a cell penetration peptide and a reprogramming factor For example, linkage may be via a biotin molecule attached, e.g., to a cell penetration peptide and streptavidin attached to the reprogramming factor polypeptide. Exemplary linkers include, but are not limited to, straight or branched-chain carbon linkers, heterocyclic carbon linkers, substituted carbon linkers, unsaturated carbon linkers, aromatic carbon linkers, peptide linkers, etc.

In embodiments where a linker is used to connect the cell penetration peptide to the reprogramming factor polypeptide, the linkers can be attached to the cell penetration peptide and/or the reprogramming factor polypeptide by any means or method known by one of skill in the art without limitation. For example, the linker can be attached to the cell penetration peptide and/or the reprogramming factor polypeptide with an

Further, portions of the transducible polypeptide to be linked, e.g., a cell penetration peptide and a reprogramming factor, can be derivatized as appropriate to facilitate linkage to another portion of the transducible polypeptide, or to a linker. Such derivatization can be accomplished, for example, by attaching a suitable derivative or derivatives such as those available from Pierce Chemical Company, Rockford, Ill. Alternatively, derivatization may involve chemical treatment of one or more portions of the transducible polypeptide to be linked, e.g., a cell penetration peptide and/or a reprogramming factor. For example, the skilled artisan can routinely generate free sulfhydryl groups on proteins to provide a reactive moiety for making a disulfide, thioether, thioester, etc. linkage. See, e.g., U.S. Pat. No. 4,659,839.

Any of the linking methods described herein can be used to link portions of transducible polypeptides, e.g., a cell penetration peptide and a reprogramming factor, in various configurations. For example, the carboxy terminus of the cell penetration peptide may be linked, directly or indirectly, to the amino terminus of the reprogramming factor polypeptide. In some embodiments, the carboxy terminus of the reprogramming factor may be linked to the amino terminus of the cell penetration peptide, either directly or indirectly. In other embodiments, the amino terminus of the cell penetration peptide may be linked, either directly or indirectly, to the amino terminus of the reprogramming factor. In other embodiments, the carboxy terminus of the cell penetration peptide may be linked, either directly or indirectly, to the carboxy terminus of the reprogramming factor. As discussed above, as used herein, “linked to” an amino terminus or a carboxy terminus does not necessarily connote a direct linkage to the amino-most, or carboxy-most amino acid of the polypeptide, but can also be via a linker, e.g., an amino acid sequence of one or more residues, e.g., 2, 3, 4, 5, 10, 15, 20, 25, or more amino acid residues.

It is noted that any transducible polypeptide made via methods described above can be utilized as part of the methods described herein.

6.2.4. Conditions for Generating Reprogrammed Cells

Any method that can contact transducible polypeptides with a cell, for example, a differentiated mammalian cell or a cell exhibiting at least one characteristic of pluripotency, can be utilized in conjunction with the methods presented herein. For example, in one embodiment, supernatants, extracts, or co-cultures of cells producing transducible polypeptides useful for the methods described herein can be used to contact transducible polypeptides to a cell. Alternatively, transducible polypeptides can be purified using standard techniques in the art, and added to a culture medium, e.g., as a medium supplement, to contact cells present in or added to the culture medium.

Cells may be contacted with a composition comprising transducible polypeptides for varying periods of time. In one embodiment, differentiated mammalian cells are contacted in vitro with the composition for a period of time sufficient to generate reprogrammed cells that exhibit at least one characteristic of pluripotency. In some embodiments the cells are contacted with the composition for a period of time between 1 hour and 30 days. For example, the period may be 1 day, 3 days, 5 days, 7 days, 10 days, 12 days, 15 days or more. In some embodiments, the period is 1, 2, 3, 4, 5, 6, 7, 8, 9, 10, 11, 12, 13, 14, or 15 days. In a particular embodiment, the period of contact is 5 days. It is contemplated that differentiated cells contacted with one or more transducible polypeptides for a particular duration of time may receive more than one, e.g., repeated, administrations of the one or more transducible polypeptides during the contact period. For example, differentiated cells can be contacted with one or more transducible polypeptides for a total period of 10, 11, 12, 13, 14 or 15 days, with culture medium comprising the transducible polypeptides being replaced every 2^{nd} 3^{th}, 4^{th} or 5^{th} day, or later.

Thus, in some embodiments, the differentiated cells are contacted with one or more transducible polypeptides for a first period of time, followed by contact with one or more transducible polypeptides for a second period of time. In some embodiments, the differentiated cells are contacted with the same transducible polypeptide or combination of transducible polypeptides for a first period of time and a second period of time. In other embodiments, the differentiated cells are contacted with a first transducible polypeptide or combination of transducible polypeptides, for a first period of time, followed by contact with a second, different transducible polypeptide or combination of transducible polypeptides for a second period of time.

In some embodiments where a differentiated cell is contacted with a combination of transducible polypeptides, the differentiated cells are contacted with each transducible polypeptide of the combination concurrently, that is, each of the transducible polypeptides is contacted to the cells or the culture containing the cells simultaneously. In some embodiments, the differentiated cells are contacted with each of the transducible polypeptides of within about 1, 2, 3, 4, 5, 6, 7, 8, 9, 10, 11 or 12 hours. In some embodiments, the differentiated cells are contacted with each of the transducible polypeptides or a subcombination of the transducible polypeptides sequentially. In some embodiments, sequential contacting of each transducible polypeptide comprises contacting the differentiated cells with a first transducible polypeptide or combination of transducible polypeptides, then contacting the differentiated cells with a second transducible polypeptide or combination of transducible polypeptides at a later time point, etc., until each transducible polypeptide of the entire combination has been contacted to the cells. In some embodiments, each transducible polypeptide or combination of transducible polypeptides is contacted about 12, 14, 16, 18, 20, 22 or 24 hours apart until each transducible polypeptide of the entire combination has been contacted to the cells. In other embodiments, each transducible polypeptide or combination of transducible polypeptides is contacted about 1, 2, 3, 4, 5, 6 or more days apart until each of the transducible polypeptides of the entire combination has been contacted to the cells.

The differentiated cells may be contacted with one or more transducible polypeptides at varying concentrations. In some embodiments, the cells are contacted with an equimolar amount of each transducible polypeptide. For example, in some embodiments, the cells are contacted with a plurality of transducible polypeptides comprising equimolar amounts of an Oct ¾ polypeptide linked to a cell penetration peptide, a Sox2 polypeptide linked to a cell penetration peptide, a Nanog polypeptide linked to a cell penetration peptide, and a Lin28 polypeptide linked to a cell penetration peptide. In other embodiments, the concentration of one or more transducible polypeptides may be differ (may be higher or lower) relative to the concentration of one or more of the other transducible polypeptides, e.g., increased or decreased to enhance the overall efficiency of generating reprogrammed cells.

In some embodiments, the differentiated cells are contacted with a concentration of between about 0.01-10, 0.1-50, 5-100, 50-100, 100-150, 150-200 μg/ml or more of total transducible polypeptides for a duration of time sufficient to reprogram the cell to exhibit at least one characteristic of pluripotency. In some embodiments, the differentiated mammalian cells are contacted with a concentration of about 0.01, 0.05, 0.1, 0.2, 0.3, 0.4, 0.5, 0.6, 0.7, 0.8, 0.9 or 1.0 μg/ml of total transducible polypeptides for a duration of time sufficient to generate a reprogrammed cell that exhibits at least one characteristic of pluripotency. In some embodiments, the differentiated mammalian cells are contacted with a concentration of about 1, 2, 3, 4, 5, 6, 7, 8, 9 or 10 μg/ml of total transducible polypeptides for a duration of time sufficient to generate reprogrammed cells that exhibit at least one characteristic of pluripotency. In some embodiments, the differentiated cells are contacted with a concentration of about 10, 15, 20, 25, 30, 35, 40, 45 or 50 μg/ml of total transducible polypeptides for a duration of time sufficient to generate reprogrammed cells that exhibit at least one characteristic of pluripotency. In some embodiments, the differentiated cells are contacted with a concentration of about 50, 75, 100, 125, 150 or 200 μg/ml of total transducible polypeptides for a duration of time sufficient to generate reprogrammed cells that exhibit at least one characteristic of pluripotency.

Cells may be maintained in culture for varying periods of time prior to assessing the cells for characteristics of pluripotency. Thus in certain methods, differentiated cells which have been contacted with one or more transducible polypeptides are maintained in culture for 1, 2, 3, 4, 5 days, or more than 5 days prior to identifying or selecting for reprogrammed cells. In some embodiments, differentiated cells contacted with one or more transducible polypeptides are maintained in culture for at least 4, 5, 6, 7, 8, 9, 10, 11, 12, 13, 14, 15, 16, 17, 18, 19, 20, 21 or more days (e.g., between 3-5 weeks) prior to identifying or selecting for reprogrammed cells.

In some embodiments, differentiated cells which have been contacted with one or more transducible polypeptides, that is, putative reprogrammed cells, are cultured using methods well known in the art, for example, by culturing on feeder cells, such as irradiated fibroblasts, or in conditioned media obtained from cultures of such feeder cells, in order to obtain continued long-term cultures of the induced pluripotent stem cells. In certain embodiments, the putative reprogrammed cells to be expanded can be exposed to, or cultured in the presence of, an agent that suppresses cellular differentiation. Such agents are well-known in the art and include, but are not limited to, human Delta 1 and human Senate 1 polypeptides (see, Sakano et al., U.S. Pat. No. 6,337,387 entitled “Differentiation suppressive polypeptide,” issued Jan. 8, 2002), leukemia inhibitory factor (LIF) and stem cell factor. Methods for the expansion of cell populations are also known in the art (see e.g., Emerson et al., U.S. Pat. No. 6,326,198 entitled “Methods and compositions for the ex vivo replication of stem cells, for the optimization of hematopoietic progenitor cell cultures, and for increasing the metabolism, GM CSF secretion and/or IL 6 secretion of human stromal cells”, issued Dec. 4, 2001; Kraus et al., U.S. Pat. No. 6,338,942, entitled “Selective expansion of target cell populations,” issued Jan. 15, 2002). In particular embodiments, the putative reprogrammed cells are cultured in the presence of LIF and Bone Morphogenetic Protein 4 (BMP4), as described by Chou et al., Cell 135:449-461 (2008).

In particular embodiments, where mouse embryonic fibroblasts have been subjected to methods provided herein, the treated fibroblasts can be plated on mitomycin C treated SNL feeder cells (a mouse cell line stably transfected with leukemia inhibitory factor (LIF) and a neomycin resistance gene) in medium designed for culture of primate embryonic stem cells supplemented with bFGF. See, Takahashi et al., Cell 131:861-72 (2007); Takahashi et al., Nat Protoc 2: 3081-9 (2007).

The putative reprogrammed cells may be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

6.2.5 Differentiated Cells

Differentiated cells that can be used in accordance with the methods provided herein can be any differentiated cells known in the art. In certain embodiments, in instances where the differentiated cells are to be administered to a subject once reprogrammed or once differentiated after reprogramming, the differentiated cells can be reprogrammed and/or differentiated ex vivo, that is, the cells to be reprogrammed and/or differentiated can be obtained from the subject, reprogrammed outside the body of the subject, and then administered back to the subject. In certain embodiments, therefore, where the differentiated cells are to be administered to a subject once reprogrammed or once differentiated after reprogramming, the can be autologous or heterologous to the subject.

The differentiated cell can be any differentiated cell of a vertebrate species. In some embodiments, the differentiated cell is a differentiated mammalian cell. In some embodiments, the differentiated cell is a differentiated cell derived from a primate. In some embodiments, the species of the differentiated cell is human, murine, e.g., mouse, porcine, bovine, canine, equine or feline.

In some embodiments, the differentiated cells are somatic cells. In some embodiments, the somatic cells are adult somatic cells. In some embodiments, the somatic cells are native somatic cells. In some embodiments, the somatic cells have been genetically engineered or altered. Suitable mammalian somatic cells can also include, but are not limited to, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, cardiac muscle cells, and other muscle cells, etc. In some embodiments, the differentiated cell is a fibroblast, e.g., an adult fibroblast or an embryonic fibroblast. In some embodiments, the differentiated cell is a cell of hematopoietic lineage. In some embodiments, the differentiated cell is derived from peripheral blood. In some embodiments, the differentiated cell is a cell of the immune system, including, but limited to, a macrophage, a lymphoid cell, an immature B cell (e.g., pro-B cell or pre-B cell), and a mature B cell (e.g., a non-naive B-cell).

In some embodiments, the somatic cells are adult stem cells, e.g., cells that are capable of giving rise to all the cell types of a particular tissue. Exemplary adult stem cells include hematopoietic stem cells, neural stem cells, cardiac stem cells, e.g., cardiosphere derived cells (CDCs; see e.g., PCT International Publication No. WO 06/052925), and mesenchymal stem cells.

In some embodiments, the differentiated cells can be primary cells, e.g., non-immortalized cells, such as those newly isolated from a subject, and the cells can be maintained in cell culture following their isolation from the subject and prior to performing a method of the invention. In some embodiments, the differentiated cells are passaged at least once or more than once prior to their use in the methods provided herein. In some embodiments, the cells are passaged between 2-10, 10-20, 20, 30, 30-40, 40-50 or more than 50 times prior to being subjected to the methods of the invention. In other embodiments the cells will have been passaged no more than 1, 2, 5, 10, 20, or 50 times prior to being Subjected to the methods of the invention.

Differentiated cells suitable for the methods provided herein can be obtained by any method known in the art, and can be obtained from any organ or tissue containing live somatic cells, e.g., blood, bone marrow, skin, lung, pancreas, liver, stomach, intestine, heart, pancreas, reproductive organs, bladder, kidney, urethra and other urinary organs, etc.

6.2.6 Characteristics of Reprogrammed Cells

The reprogrammed cells generated by the methods provided herein exhibit at least one characteristic of pluripotency. In some embodiments, the reprogrammed cell displays at least one characteristic of pluripotency if the cell: expresses at least one marker, as detected by RT-PCR or cell sorting techniques, of pluripotency selected from the group consisting of SSEA-3, SSEA-4, TRA-1-60, Nanog, and Oct ¾; displays the ability to form embryoid bodies in vitro; can form tightly packed colonies on culture plates; displays the ability to differentiate into cells having characteristics of endoderm, mesoderm or ectoderm when injected into SCID mice; displays the ability to form teratomas if injected into animals; exhibits an exponential pattern of growth in cell culture, without senescence; exhibits telomerase activity; exhibits the ability to undergo at least between 10-40 population doublings in culture; comprises unmethylated DNA characteristic of pluripotent clones; or displays germline competence. It is noted that a cell that exhibits at least one characteristic of pluripotency can include, for example, a multipotent cell or a pluripotent cell. It is further noted, in the context of reprogrammed cells, that in instances whereby the differentiated mammalian cell used in generating the reprogrammed cell itself exhibits at least one characteristic of pluripotency, the resulting reprogrammed mammalian cell exhibits at least one additional characteristic of pluripotency relative to the differentiated mammalian cell, and/or exhibits quantitatively more of at least one characteristic of pluripotency relative to the differentiated mammalian cell.

Determination that a reprogrammed cell has been generated that displays at least one characteristic of pluripotency may be accomplished by methods well-known in the art, e.g., measuring changes in morphology and cell surface markers using techniques such as flow cytometry or immunocytochemistry (e.g., staining cells with tissue-specific or cell-marker specific antibodies), by examination of the morphology of cells using light or confocal microscopy, or by measuring changes in gene expression using techniques well known in the art, such as PCR and gene-expression profiling.

In some embodiments, differentiated cells subjected to the methods provided herein can be characterized by examining the expression of genes which are normally expressed in undifferentiated cells and are indicative of a pluripotent state. For example, the gene expression pattern of the reprogrammed cells may be compared to the gene expression pattern of embryonic cells or other undifferentiated cells. In some embodiments, the embryonic or undifferentiated cell to which the putative reprogrammed cell is compared to is from the same species as the original differentiated cell subjected to reprogramming. In other embodiments, the absence of the expression of genes normally associated with a differentiated state can be examined to monitor the extent of reprogramming.

In some embodiments, a reprogrammed cell that exhibits at least one characteristic of pluripotency can be identified by the presence of anyone of the following cell surface markers: SSEA3, SSAE4, Nanog, Sox2 and Tra-1-60. In some embodiments, such cells can be identified by the presence of at least two, three, or all four of these cell surface markers.

Expression of such cell surface markers are routinely determined according to methods well known in the art, e.g. by flow cytometry, followed by washing and staining with an anti-cell surface marker antibody. For example, to determine the presence of SSEA-3 or SSEA-4, cells may be washed in PBS and then double-stained with an anti-SSEA-3 antibody labeled with, for instance, phycoerythrin, and an anti-SSEA-4 antibody labeled with, for instance, fluorescein isothiocyanate. Other techniques known in the art for examining protein expression, e.g., immunofluorescence microscopy, Western blot, protein microarrays, and the like, can be used to determine the presence of the cell surface markers. RT-PCR can also be used to assess mRNA expression of SSEA3, SSAE4, Nanog, Sox2 and Tra-1-60 in differentiated cells subjected to the methods provided herein.

In some embodiments, a reprogrammed cell that exhibits at least one characteristic of pluripotency can be identified based on the expression of a reporter construct within the cells, wherein a reporter gene, e.g., green fluorescent protein (GFP), is operably linked to a promoter sequence of a gene, the expression of which is typically associated with a state of pluripotency, such as SSEA3, SSEA4, Nanog, Sox2, or Tral-60. In some embodiments, the reporter construct comprises GFP under the control of a Nanog promoter (SYSTEM BIOSCIENCE, Inc. CA). See Yamanaka et al., Nature 448:313-7 (2007).

In some embodiments, a reprogrammed cell that exhibits at least one characteristic of pluripotency can be identified by evaluating certain morphological criteria with reference to the morphological characteristics of an embryonic stem (ES) cell or an ES cell colony. In some embodiments, the morphological criteria includes any visually detectable aspect of the size, shape, structure, organization, and/or physical form of the putative reprogrammed cells or colonies. Morphological criteria include, e.g., the shape of the colonies, the sharpness of colony boundaries (with sharp boundaries characterizing colonies of ES-like cells), the density of the cells in the colonies (with increased density characterizing colonies of ES-like cells), and/or the small size and distinct shape of the putative reprogrammed cells relative to naive differentiated cells.

### 6.3 Methods of Using Reprogrammed Cells

Reprogrammed cells can be used for a variety of purposes. For example, reprogrammed cells can be utilized for cell transplantation and cell therapy, can be used to generate one or more differentiated cell types, and can be useful in the treatment of diseases or disorders, including, but not limited to, vascular disease, neurological diseases or disorders, autoimmune diseases or disorders, diseases or disorders involving inflammation, and cancer or the disorders associated therewith. In one embodiment, the populations of reprogrammed cells are used to renovate and repopulate tissues and organs, thereby replacing or repairing diseased tissues, organs or portions thereof.

In some embodiments, the methods provided herein generate induced pluripotent stem cells, and thus provide an important advance in the art of creating inducible pluripotent stem cells. For example, since differentiated cells are contacted with reprogramming factors which are provided in the form of recombinant transducible polypeptides, the methods obviate the need for viral transduction or plasmid transfection of the cell, thereby eliminating the possibility of permanent genetic modification of the cell with oncogenic or immortalizing gene sequences. This alleviates concerns over the prolonged exogenous expression of pluripotency genes which have been shown to also have oncogenic potential, e.g., c-myc.

6.3.1 Differentiation

In certain embodiments, differentiated cell types are derived from the reprogrammed cell generated by the methods provided herein. For example, differentiated cells may be obtained by culturing reprogrammed cells in the presence of at least one differentiation factor and selecting differentiated cells from culture. Selection of differentiated cells may be based on phenotype, such as the expression of particular cell markers normally present on differentiated cells. Alternatively, functional assays which screen for the performance of one or more functions associated with a particular differentiated cell type may be performed.

Accordingly, reprogrammed cells generated by the methods provided herein may be differentiated into any of the cells in the body including, without limitation, skin, cartilage, bone skeletal muscle, cardiac muscle, renal, hepatic, blood and blood forming, vascular precursor and vascular endothelial, pancreatic beta, neurons, glia, retinal, inner ear follicle, intestinal, lung, cells.

In another aspect, provided herein are isolated populations of cells comprising at least 70%, at least 80%, at 90%, or at least 95% cells that have been differentiated using reprogrammed cells generated by the methods provided herein. In one particular embodiment, the cells that have been differentiated are cardiac cells. Also provided herein are such isolated populations of cells further comprising at least one other isolated population of cells, e.g., reprogrammed cells generated via the methods provided herein, stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix. In one embodiment, the isolated population of cells comprises about 1×10⁵, 5×10⁵, 1×10⁶, 5×10⁶, 1×10⁷, 5×10⁷, 1×10⁸, or 5×10⁸ cells, either cells that have been differentiated using reprogrammed cells generated by the methods provided herein or total cells. In one embodiment, the isolated population of cells is present in a formulation suitable for administration to a human. In another embodiment, the isolated population of cells is present in a bag, e.g., a plastic bag, such as a plastic bag suitable for use in administration of the cells to a human. In another embodiment, the isolated population of cells is present in a syringe, such as a sterile syringe suitable for administration of the cells to a human. In yet another embodiment, these cells are present on a solid support, e.g., a scaffold or matrix, such as a synthetic matrix or previously decellularized matrix, for example, a three-dimensional scaffold or matrix.

In another embodiment, the reprogrammed cells are exposed to inducers of differentiation to yield other therapeutically useful cells, such as retinal pigment epithelium, definitive endoderm, pancreatic beta cells and precursors to pancreatic beta cells, hematopoietic precursors and hemangioblastic progenitors, neurons, respiratory cells, muscle progenitors, cartilage and bone-forming cells, cells of the inner ear, neural crest cells and their derivatives, gastrointestinal cells, liver cells, kidney cells, smooth and cardiac muscle cells, dermal progenitors including those with a prenatal pattern of gene expression useful in promoting scarless wound repair, as well as many other useful cell types of the endoderm, mesoderm, and endoderm. Such inducers include but are not limited to: cytokines such as interleukin-alpha A, interferon-alpha AID, interferon-beta, interferon-gamma, interferon-gamma-inducible protein-10, interleukin-I-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6kine, activin A, amphiregulin, angiogenin, B-endothelial cell growth factor, beta ceellulin, brain-derived neurotrophic factor, ClO, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FL T-3/FLK-2 ligand, glial cell line-derived neurotrophic factor, Gly-His-Lys, granulocyte colony stimulating factor, granulocytemacrophage colony stimulating factor, GRO-alpha/MGSA, GRO-beta, GRO-gamma, HCC-I, heparin-binding epidermal growth factor, hepatocyte growth factor, heregulin-alpha, insulin, insulin growth factor binding protein-l, insulin-like growth factor binding protein-1, insulin-like growth factor, insulin-like growth factor II, nerve growth factor, neurotophin-3, 4, oncostatin M, placenta growth factor, pleiotrophin, rantes, stem cell factor, stromal cell-derived factor IB, thromopoietin, transforming growth factor-(alpha, beta 1,2,3,4,5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine/leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopres sin, extracellular matrix components such as fibronectin, proteolytic fragments of fibronectin, laminin, tenascin, thrombospondin, and proteoglycans such as aggrecan, heparin sulphate proteoglycan, chondroitin sulphate proteoglycan, and syndecan. Other inducers include cells or components derived from cells from defined tissues used to provide inductive signals to the differentiating cells derived from the reprogrammed cells of the invention. Such inducer cells may derive from human, nonhuman mammal, or avian, such as specific pathogen-free (SPF) embryonic or adult cells.

In a particular embodiment, the reprogrammed cells are induced to undergo cardiac differentiation by sequentially exposing the cells to Activin A and Bone Morphogenic Protein-4 (BMP4), followed by Percoll centrifugation, as described by Laflamme et al., Nat Biotechnology 25:1015-24 (2007); and Schuldiner et al., Proc Natl Acad Sci USA 97:11207-12 (2007), the contents of which are incorporated herein in their entireties.

In another particular embodiment, the reprogrammed cells are induced to undergo cardiac differentiation by sequentially exposing the cells to Activin A and Bone Morphogenic Protein-4 (BMP4), followed by Percoll centrifugation, then enriching for cells that express flk-1 and CXCR4 biomarkers, as described by Yang et al., Nature 453:524-8 (2008); and Nelson et al., Stem Cells 26:1464-73 (2008), the contents of which are incorporated herein in their entireties. The CXCR4/FIk-1 biomarker pair predicts the emergence of cardiogenic specification within a pluripotent stem cell pool, thus enabling the targeted selection of cells having a cardiopoietic lineage. Cells expressing flk-1 and CXCR4 markers can be routinely determined according to methods well known in the art, e.g. by flow cytometry.

In another particular embodiment, the reprogrammed cells are induced to undergo cardiac differentiation by exposing the cells to dickkopf homolog 1 (Dkk-1).

In another aspect, provided herein are isolated populations of cells comprising at least 70%, at least 80%, at 90%, or at least 95% cells that have been differentiated using reprogrammed cells generated by the methods provided herein. In one particular embodiment, the cells that have been differentiated are cardiac cells. Also provided herein are such isolated populations of cells further comprising at least one other isolated population of cells, e.g., reprogrammed cells generated via the methods provided herein, stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In one embodiment, the isolated population of cells comprises about 1×10⁵, 5×10⁵, 1×10⁶, 5×10⁶, 1×10⁷, 5×10⁷, 1×10⁸, or 5×10⁸ cells, either cells that have been differentiated/using reprogrammed cells generated by the methods provided herein or total cells. In one embodiment, the isolated population of cells is present in a formulation suitable for administration to a human. In another embodiment, the isolated population of cells is present in a bag, e.g., a plastic bag, such as a plastic bag suitable for use in administration of the cells to a human. In another embodiment, the isolated population of cells is present in a syringe, such as a sterile syringe suitable for administration of the cells to a human. In yet another embodiment, these cells are present on a solid support, e.g., a scaffold or matrix, such as a synthetic matrix or previously decellularized matrix, for example, a three-dimensional scaffold or matrix.

### 6.4 Cardiac Differentiation

In another aspect, provided herein are methods for inducing differentiation of a mammalian cell that exhibits at least one characteristic of pluripotency, e.g., a pluripotent or multipotent cell, towards a cardiac lineage. The cells include, but are not limited to, reprogrammed cells generated by the methods provided herein, embryonic stem cells, embryonic-like stem cells, cardiac stem cells, e.g., cardiosphere derived cells, and induced pluripotent stem cells. In a particular embodiment, reprogrammed cells generated by the methods provided herein can be induced to differentiate into cardiac cells.

Mammalian cells exhibiting cardiac differentiation generated via methods provided herein can be utilized in treatment of cardiac disorders and/or in amelioration of symptoms relating to cardiac disorders such as cardiac ischemia, myocardial infarction, and heart failure, including congestive heart failure.

In a particular embodiment, the cells are induced to undergo cardiac differentiation by contacting the cells with a transcription factor capable of inducing cardiac differentiation. In some embodiments, the transcription factor is provided in the form of a transducible polypeptide comprising a cell penetration peptide as described herein, linked to the amino acid sequence of the transcription factor. Thus, in certain embodiments, cells, e.g., induced pluripotent stem cells, can be induced to undergo cardiac differentiation, such that the cells exhibit at least one characteristic of cardiac differentiation as described herein, by contacting the cells with transducible polypeptides comprising a cell penetration peptide, e.g., herpes viral VP22 protein, HIV-1 TAT, AntP HD, or poly-arginine, and a transcription factor having cardiac-differentiating activity for a time sufficient to induce cardiac differentiation of the cell, that is, to generate a cell that exhibits at least one characteristic of a cardiac cell.

In certain embodiments, the transcription factor having cardiac-differentiating activity is islet 1 (Isl 1). The sequences of human and mouse Isl 1 have been described previously. See, e.g., Roose et al., Genomics 57 (2), 301-305 (1999); Karlsson et al., Nature 344 (6269), 879-882 (1990). Representative cDNA and amino acid sequences of human Isl 1 are provided herein as SEQ ID NOS: 37 and 38, respectively.

Thus, in one aspect, provided herein is a method for cardiac differentiation of a mammalian cell exhibiting at least one characteristic of pluripotency comprising: contacting the mammalian cell with a transducible polypeptide so that cardiac differentiation of the mammalian cell occurs. In some embodiments, the transducible polypeptide comprises an islet 1 (ISL 1) polypeptide linked to a cell penetration peptide. In some embodiments, the transducible polypeptide comprises a human ISL 1 polypeptide. In some embodiments, the cell penetration peptide comprises HIV-I TAT. In some embodiments, the cell penetration peptide comprises AntP HD. In some embodiments, the cell penetration peptide comprises poly-arginine. In a particular embodiment, the cell penetration peptide comprises herpes viral VP22 protein.

In one embodiment, provided herein is a method for cardiac differentiation of a mammalian cell exhibiting at least one characteristic of pluripotency comprising: contacting the mammalian cell in vitro, for example, ex vivo, with a transducible polypeptide so that cardiac differentiation of the mammalian cell occurs. In some embodiments, the transducible polypeptide comprises an islet 1 (ISL 1) polypeptide linked to a cell penetration peptide. In some embodiments, the transducible polypeptide comprises a human ISL I polypeptide. In some embodiments, the cell penetration peptide comprises HIV-1 TAT. In some embodiments, the cell penetration peptide comprises AntP HD. In some embodiments, the cell penetration peptide comprises poly-arginine. In a particular embodiment, the cell penetration peptide comprises herpes viral VP22 protein.

In a particular embodiment, the transducible polypeptide comprises the carboxy terminus of VP22 linked to the amino terminus of Isl 1, such as the transducible polypeptide provided herein as SEQ ID NO:39. In another particular embodiment, the transducible polypeptide comprises the carboxy terminus of Isl 1 linked to the amino terminus of VP22, such as the transducible polypeptide provided herein as SEQ ID NO:40. The linkage can be direct or indirect.

In other embodiments, the transcription factor having cardiac-differentiating activity is selected from the group consisting of ISL 1, GATA-4, MEF2C, Nkx2.5, Hand-1, Hand-2, TBX5 and Twist-1, e.g., human ISL1, GATA-4, MEF2C, Nkx2.5, Hand-I, Hand-2, TBX5 and Twist-1. Accordingly, in certain aspects, provided herein is a method for cardiac differentiation of a mammalian cell exhibiting at least one characteristic of pluripotency comprising: contacting the mammalian cell with one or more transducible polypeptides so that cardiac differentiation of the mammalian cell occurs, wherein the transducible polypeptide comprises an ISL 1 polypeptide linked to a cell penetration peptide, GATA-4 polypeptide linked to a cell penetration peptide, a MEF2C polypeptide linked to a cell penetration peptide, a Nkx2.5 polypeptide linked to a cell penetration peptide, a Hand-1 polypeptide linked to a cell penetration peptide, a Hand-2 polypeptide linked to a cell penetration peptide, a TBX5 polypeptide linked to a cell penetration peptide, or a Twist-1 polypeptide linked to a cell penetration peptide. In certain embodiments, the mammalian cell exhibiting at least one aspect of pluripotency is contacted in vitro, for example ex vivo.

In some embodiments, the differentiated mammalian cell is contacted with at least 2, 3, 4, 5, 6, 7 or 8 different transducible polypeptides selected from the group consisting of an ISL 1 polypeptide linked to a cell penetration peptide, GATA-4 polypeptide linked to a cell penetration peptide, a MEF2C polypeptide linked to a cell penetration peptide, a Nkx2.5 polypeptide linked to a cell penetration peptide, a Hand-1 polypeptide linked to a cell penetration peptide, a Hand-2 polypeptide linked to a cell penetration peptide, a TBX5 polypeptide linked to a cell penetration peptide, or a Twist-1 polypeptide linked to a cell penetration peptide. In some embodiments, the cell penetration peptide is selected from the group consisting of VP22, TAT, AntP HD, poly-arginine, or transducing fragments thereof.

As provided herein, a transducible polypeptide comprising a transcription factor having activity, e.g., ISL 1, linked to a cell penetration peptide can be made using any of a variety of methods well known to those of skill in the art, including the methods of making a transducible peptide described in Section 5.2.3 above. In some embodiments, transcription factor having cardiac-differentiating activity is linked to the cell penetration peptide via a peptide bond. In some embodiments, a transducible polypeptide comprising a transcription factor having cardiac-differentiating activity linked to a cell penetration peptide may optionally comprise a nuclear localization signal, e.g., PKKKRKV (SEQ 10 NO:37) of SV 40 large T antigen, to enhance nuclear localization of the peptide. In some embodiments, a transducible polypeptide comprising a transcription factor having cardiac-differentiating activity linked to a cell penetration peptide may optionally comprise a purification moiety, e.g., a polyhistidine moiety, to facilitate isolation and purification of the transducible peptide.

Any of the linking configurations or methods described in Section 5.2.3 may be used to link a transcription factor having cardiac-differentiating activity to a cell penetration peptide in a variety of configurations. For example, the carboxy terminus of the cell penetration peptide may be linked, directly or indirectly, to the amino terminus of the cardiac-differentiating factor polypeptide. In some embodiments, the carboxy terminus of the cardiac-differentiating factor may be linked to the amino terminus of the cell penetration peptide, either directly or indirectly. In other embodiments, the amino terminus of the cell penetration peptide may be linked, either directly or indirectly, to the amino terminus of the cardiac-differentiating factor. In other embodiments, the carboxy terminus of the cell penetration peptide may be linked, either directly or indirectly, to the carboxy terminus of the cardiac-differentiating factor.

In another aspect, provided herein is an isolated mammalian cell that exhibits at least one characteristic of cardiac differentiation, wherein the isolated mammalian cell is generated by a method provided herein.

In one embodiment, provided herein is an isolated mammalian cells that exhibits at least one characteristic of cardiac differentiation, wherein the isolated mammalian cell is generated via a method comprising: contacting a mammalian cell that exhibits at least one characteristic of pluripotency, for example, contacting the in vitro, for example ex vivo, with a transducible polypeptide so that cardiac differentiation of the mammalian cell occurs, wherein the transducible polypeptide comprises an islet 1 (ISL1) polypeptide linked to a cell penetration peptide.

In another aspect, the invention provides an isolated population of cells comprising at least 70%, 80%, 90%, 95% or 98% cells that exhibit at least one characteristic of cardiac differentiation, wherein the isolated cells are generated by a method provided herein. Also provided herein are such isolated populations of cells further comprising at least one other isolated population of cells, e.g., reprogrammed cells generated via the methods provided herein, stem cells, for example hematopoietic stem cells, stromal cells and/or isolated differentiated cells, for example adult cells. Further provided are such isolated populations of cells present on a solid support, e.g., a scaffold or matrix, such as a synthetic or previously decellularized matrix, for example, a three-dimensional scaffold or matrix.

In one embodiment, the isolated population of cells comprises about 1×10⁵, 5×10⁵, 1×10⁶, 5×10⁶, 1×10⁷, 5×10⁷, 1×10⁸, or 5×10⁸ cells, either cells that exhibit at least one characteristic of cardiac differentiation have been generated by the methods provided herein, or total cells. In one embodiment, the isolated population of cells is present in a formulation suitable for administration to a human. In another embodiment, the isolated population of cells is present in a bag, e.g., a plastic bag, such as a plastic bag suitable for use in administration of the cells to a human. In another embodiment, the isolated population of cells is present in a syringe, such as a sterile syringe suitable for administration of the cells to a human. In yet another embodiment, these cells are present on a solid support, e.g., a scaffold or matrix, such as a synthetic matrix or previously decellularized matrix, for example, a three-dimensional scaffold or matrix.

In another aspect, the invention provides an isolated population of cells comprising at least 70%, 80%, 90%, 95% or 98% cells that exhibit at least one characteristic of cardiac differentiation, wherein the isolated cells are generated by a method comprising: contacting a mammalian cell that exhibits at least one characteristic of pluripotency, e.g., contacting the cell in vitro, for example, ex vivo, with a transducible polypeptide so that cardiac differentiation of the mammalian cell occurs, wherein the transducible polypeptide comprises an islet 1 (ISL1) polypeptide linked to a cell penetration peptide.

The cells generated by the methods provided in this section exhibit at least one characteristic of cardiac differentiation. In some embodiments, the cell displays at least one characteristic of cardiac differentiation if the cell: expresses at least one marker of cardiac differentiation selected from the group consisting of a-myosin heavy chain protein, natriuretic precursor A (ANP), ryanodine receptor and SERCA; forms sarcomeres in culture; appears visibly to be visibly beating in vitro; or demonstrates spontaneous membrane depolarization.

An exemplary method for cardiac differentiation of a pluripotent mammalian cell comprising contacting the cell with a transducible polypeptide comprising ISL 1 and the cell penetration peptide VP22 is provided in the example of Section 6.2.

### 7. EXAMPLES

The invention is illustrated by the following examples which are not intended to be limiting in any way.

### 7.1 Example 1

### Reprogramming of Human Adult Fibroblasts Using Transducible Polypeptides

This example provides an exemplary method for generating a reprogrammed cell that exhibits at least one characteristic of pluripotency from a differentiated somatic cell using a plurality of different transducible polypeptides.

Primary cultures of human adult fibroblast cells are contacted in culture with a plurality of transducible polypeptides at a concentration of 0.01 mg/ml, wherein the different transducible polypeptides comprise: i) an Oct ¾ polypeptide linked to a cell penetration peptide (e.g., SEQ ID NO: 25 or SEQ ID NO: 26), ii) a Sox2 polypeptide linked to a cell penetration peptide (e.g., SEQ ID NO: 27 or SEQ ID NO: 28), iii) a c-myc polypeptide linked to a cell penetration peptide (e.g., SEQ ID NO: 33 or SEQ ID NO: 34), iv) a Klf4 polypeptide linked to a cell penetration peptide (e.g., SEQ ID NO: 35 or SEQ ID NO: 36)e, v) a Nanog polypeptide linked to a cell penetration peptide (e.g., SEQ ID NO: 29 or SEQ ID NO: 30), and vi) a Lin28 polypeptide linked to a cell penetration peptide (e.g., SEQ 10 NO: 31 or SEQ ID NO: 32). The cells are contacted with the transducible polypeptides for a two-week period, wherein cell media containing the transducible polypeptides is replaced every 2-3 days with fresh media comprising about 0.01 mg/ml of total transducible polypeptides.

The treated fibroblasts are plated on mitomycin C treated SNL feeder cells (a mouse cell line stably transduced with leukemia inhibitory factor (LIF) and a neomycin resistance gene) in medium designed for culture of primate embryonic stem cells, and supplemented with bFGF2. Three to four weeks later, putative reprogrammed cell clones are identified on the basis of their morphology (flat, tightly packed colonies). These colonies are picked and plated onto a new SNL feed cell layer, and expanded. These cells are then characterized and screened for at least one characteristic of pluripotency using techniques well known in the art, such as those described herein.

### 7.2 Example 2

### Transducible Polypeptides Comprising ISL1 Promotes Cardiac Differentiation of ES cells in vitro

7.2.1 Materials and Methods

Mouse Myocardial Infarction Model

Male C57B1/6 mice 22-28 g (Jackson Laboratory) underwent anesthesia, analgesia, tracheal intubation, pulmonary ventilation (2 cm H₂0 pressure, 120 min⁻¹, IITC Life Science, Woodland Hills, Calif.), intercostal thoracotomy and ligation of the left anterior descending (LAD) coronary artery (7-0 monofilament suture, Ethicon) to create experimental myocardial infarction. A sham surgery control group, underwent all procedures described except ligation of the LAD. ECG and rectal temperature were monitored intra-operatively. The animals were recovered overnight in a 37° C. environment. The surgeries were performed as part of an institutionally approved protocol. The animals were euthanized at 2, 7 or 14 days, (n=5 for MI and sham groups, at each time point) for harvest of cardiac tissue.

Extraction and Quantification of mRNA from Infarcted Cardiac Tissue

The infarct and peri-infarct tissue was identified by macroscopic examination and dissected out for analysis. RNA was extracted by routine laboratory methods (Trizol®, Invitrogen, CA). RNA was reverse-transcribed (iScript cDNA Synthesis Kit, Bio-Rad) and quantitative PCR performed with appropriate primers (QantiFast SYBR green PCR kit, Qiagen, CA; iCycler thermal cycler and detection software, Bio-Rad, CA). The relative abundance of the target genes was calculated by the 2^{-ΔΔCT} method (Livak et al., Methods 25:402-8 (2008)) and normalized to an internal control (GAPDH expression).

Extraction and Quantification of Protein from Infarcted Cardiac Tissue

Hearts were cut into small pieces and placed in a solution of 1% SDS and 5 mM EDTA with PBS containing protease inhibitors (BO Pharmingen, CA; cat #554779). The lysate was homogenized in lysis buffer (2% SOS, 10 mM EDTA-Na) and subjected to Western blotting by standard laboratory techniques [12% polyacrylamide gel; PVDF membrane (Immobilon P, Millipore, Mass.); primary antibodies isl1 (cat #ab20670; Abcam, CA) and c-kit (cat #AF1356; R&D systems, MN); HPO conjugated secondary antibody (cat #711-036-152, Jackson Immunoresearch, PA), chemiluminescent substrate detection (cat #34077; Pierce, Ill.) and light film (BioMax cat #876 1520, Kodak)]. To normalize for loading conditions, the membrane was stripped at 60° C. in buffer (2% SDS, 62.5 mM Tris, 100 mM beta-mercaptoethanol), then blocked again and incubated with anti-beta-tubulin antibody (Lab Vision, CA). Quantification of protein bands was performed by densitometry with NIH ImageJ software.

Immuno-Fluorescence Microscopy

Cardiac tissue was stained for Isl 1 (primary antibody, cat #ab20670, Abcam, CA; secondary anti-mouse PITC, cat #555988, BD Biosciences, CA), and c-kit (primary antibody, cat #AF1356, R&D systems, MN; secondary antibody anti-goat TRITC, cat #705-026-147, Jackson Immunoresearch, PA).

Synthesis of Recombinant VP22-Isl 1 protein

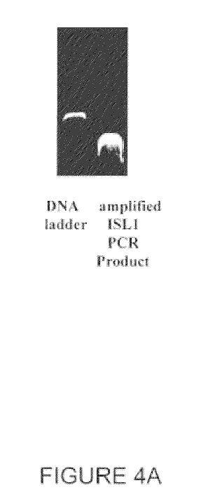

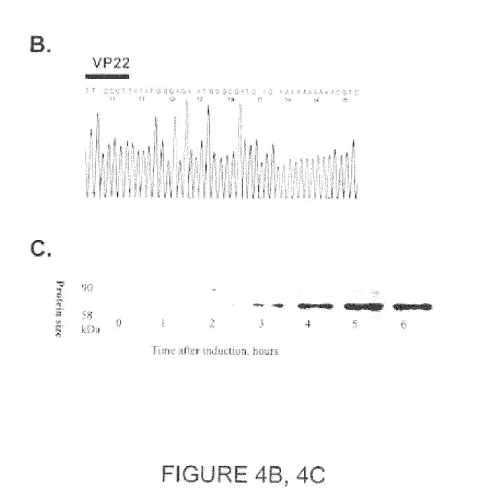

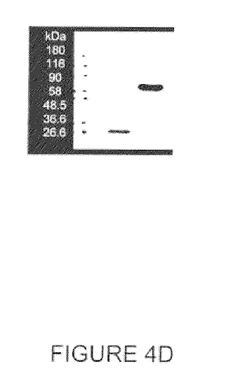

Isl 1-VP22 recombinant protein was synthesized by inserting the Isl 1 gene in frame with VP22 in the plasmid pCR®T7/VP-22-1-TOPO®, then expressing the hybrid gene product in E. coli (Voyager™ protein production kit, cat #K4860-01, Invitrogen, CA). VP22 is a structural protein of herpes simplex virus that translocates to the nucleus of mammalian cells. In brief, the method involved PCR amplification of a mouse Isl 1 cDNA clone (Open Biosystems Catalog #: EMM1002-99258597) (FIG. 4A), and ligation of the PCR product into the plasmid by the topo-isomerase enzyme. Plasmid DNA was sequenced to confirm that the Isl 2 gene was inserted in the correct orientation, and in frame with VP22 (FIG. 4B). The resulting plasmid construct was transformed into BL21 (DE3)pLysS E. Coli and expression of the T7-regulated hybrid gene was induced by isopropyl β-D-thiogalactoside (IPTG) (FIG. 4C). The expressed protein was purified by binding of His ×6 amino acid residues to a nickel resin column (ProBond™ purification, cat #K850-01, Invitrogen, CA) followed by elution. Molecular weights of purified VP22 and ISL1-VP22 proteins were confirmed by Western Blot (FIG. 40).

Mouse Embryonic Stem (ES) Cell Culture and Differentiation

RI mouse ES cells (Cat #SCRC-1036; ATCC, VA) were maintained in collagen-coated flasks, without a feeder layer, in medium containing leukemia-inhibitory factor (LIF-ESGRO™, Millipore, Mass.). The ES cells were passaged daily to prevent overcrowding of colonies. Embryoid bodies (EBs) were generated by harvesting ES cells (0.5% trypsin-EDTA, cat #59417C, Sigma-Aldrich), and culturing 10⁶ cells in 1.5 mL of medium without LIF in ultra-low attachment plates. Medium was refreshed on alternate days. After 7 days, EBs were plated onto collagen-coated dishes. On day 7, 9, 13 and 17 after withdrawal of LIF from the medium, the dishes were placed onto a 1 cm grid and the EBs inspected for beating activity. The cells were treated daily from day 1 to 7, with 10 μg of VP22-Isl 1 recombinant protein in 1.5 mL medium, and on days 8 to 10, with 30 μg of VP22-Isl 1 in 5 mL medium). The two control groups were (1) the same quantity of VP22 protein added to the medium, and (2) medium alone.

7.2.2 Results

Increased Cardiac Expression of Isl 1 by c-Kit Positive Cells Following Myocardial Infarction

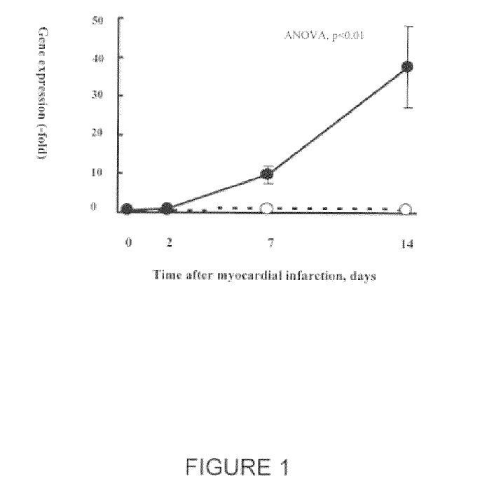

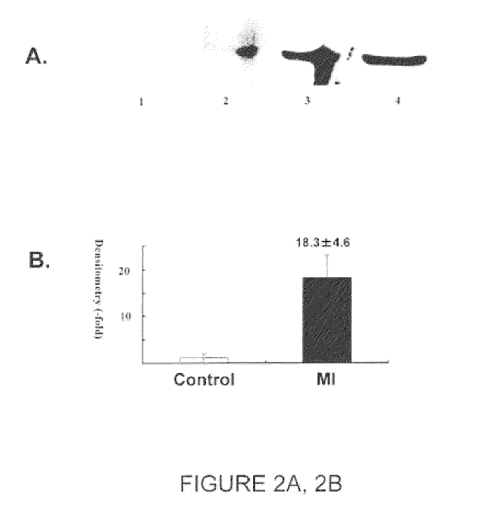

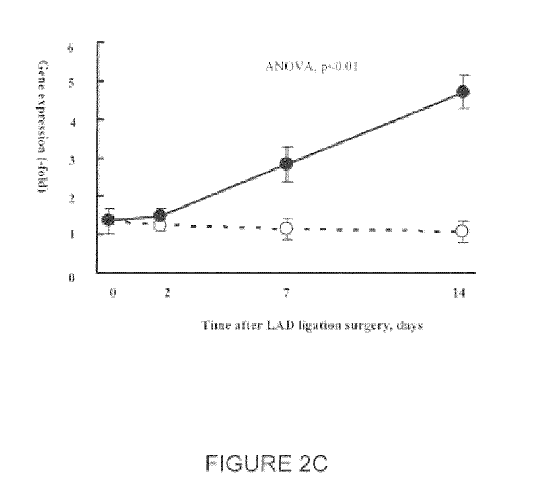

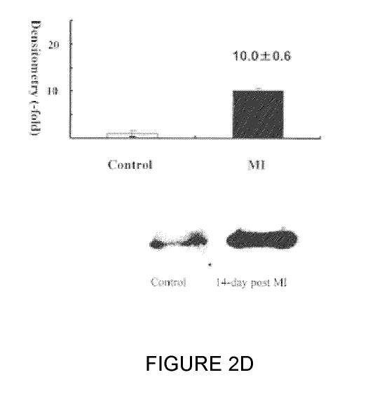

Successful creation of myocardial infarction was confirmed by upregulation of collagen gene expression at 14 days (>20 fold, n=5, p<0.01) and by histology (see FIG. 1). Two weeks following myocardial infarction, Isl 1 protein was significantly increased in infarcted and peri-infarct cardiac tissue (18.3±4.6 fold, p<0.01, FIGS. 2A and 2B), accompanied by an increase in Isl 1 mRNA expression compared to baseline (FIG. 2C).

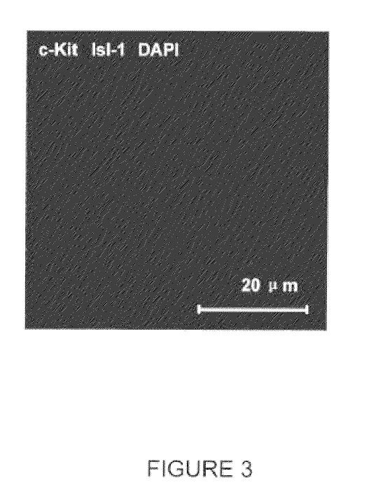

Similarly, levels of c-kit were increased in day 14 myocardial infarction tissue compared to baseline (10.0±0.6 fold, p<0.05, FIG. 2D). Immunofluorescence microscopy demonstrated co-localization of Isl 1 and c-kit in the same cells, indicating that the source of Isl 1 within the infarction tissue is a subset of c-kit positive cells (FIG. 3).

Nuclear Targeted Isl 1 Promotes Cardiac Differentiation

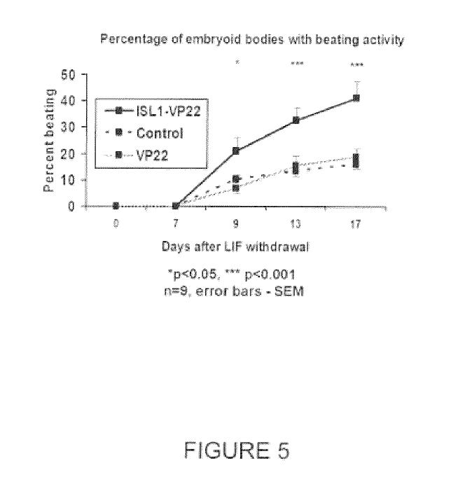

Given the finding of increased Isl 1 within recently infarcted cardiac tissue, a determination was made as to whether Isl 1 encouraged cardiogenic differentiation in mouse ES cells. Mouse ES cells (R1 cell line) were differentiated into embryoid bodies by withdrawal of LIF from the culture medium in low-attachment wells. The cells were treated with VP22-Isl 1 recombinant protein from day 1 to 10. VP22-lsll treatment markedly increased the proportion of EBs with visible beating activity compared to non-treated, or VP22 protein treated control groups (n=9) at day 9 (VP22-Isl 1: 20.9±5.4%, control: 10.3±2.1%, VP22 control: 6.6±4.5%, ANOVA P=0.02, VP22-Isl 1 compared to both control groups (LSD) p<0.05), at day 13 (VP22-Isl 1: 32.7±14.8%, control: 13.6±5.9%, VP22 control: 15.5±10.4%, ANOVA P<0.01, VP22-Isll compared to both control groups (LSD) p<0.01), and at day 17 (VP22-Isl1: 41.1±18.5%, control: 16.1±4.2%, VP22 control: 18.8±10.4%, ANOVA P<0.001, VP22-Isl 1 compared to both control groups (LSD) p<0.001). The two control groups (untreated and VP22 treated) were statistically indistinguishable from each other (FIG. 5).

7.2.3 Conclusion

The results demonstrate that the embryonic transcription factor Isl 1 is reexpressed in injured adult cardiac tissue. Furthermore, the source of expression of Isl 1 within the infarcted cardiac tissue is demonstrated to be a subset of c-kit positive cells within the tissue. These results further demonstrate that a form of the Isl 1 protein specifically engineered to localize to the nucleus clearly promotes cardiac differentiation of ES cells in vitro.

These results also demonstrate that a transducible polypeptide can be used in accordance with the present methods to modulate the differentiation state of the cell. These results also indicate that cell penetration peptides can be used to effectively introduce nuclear factors that can modulate the potency state of the cell, thereby obviating the use of viral vectors for cell programming or reprogramming.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1.-52. (canceled)

53. A method for inducing the cardiac differentiation of a population of mammalian cells comprising:
contacting said mammalian cells with a transducible peptide comprising an ISL1 polypeptide linked to a cell penetration peptide,
wherein said contacting is for a period of time sufficient to induce said mammalian cells to exhibit at least one characteristic of cardiac differentiation,
wherein prior to said contacting said mammalian cells did not exhibit at least one characteristic of cardiac differentiation,
wherein said mammalian cells exhibit at least one characteristic of cardiac differentiation if said mammalian cells:
a) express at least one marker of cardiac differentiation selected from the group consisting of alpha-myosin heavy chain protein, natriuretic precursor A (ANP), ryanodine receptor, and SERCA,
b) form sarcomeres in culture,
c) appear to be visibly beating, or
d) demonstrate spontaneous membrane depolarization;
thereby inducing the cardiac differentiation of a population of mammalian cells.

54. The method of claim 53, wherein said cell penetration peptide is selected from the group consisting of herpes viral VP22, HIV-I TAT, the homeodomain of the Drosophila melanogaster protein Antennapedia (Antp HD), poly-arginine, and transducing fragments of any of the preceding.

55. The method of claim 53, wherein said ISL1 polypeptide is linked to said cell penetration peptide via a peptide bond.

56. The method of claim 54, wherein said cell penetration peptide comprises herpes viral VP22.

57. The method of claim 56, wherein the amino terminus of ISL1 is linked to the carboxy terminus of VP22.

58. The method of claim 56, wherein the carboxy terminus of ISL1 is linked to the amino terminus of VP22.

59. The method of claim 53, wherein said induction of exhibition of at least one characteristic of cardiac differentiation is suitable for amelioration of symptoms relating to a cardiac disorder.

60. The method of claim 59, wherein said cardiac disorder is selected from the group consisting of cardiac ischemia, myocardial infarction, heart failure, and congestive heart failure.

61. The method of claim 53, wherein said mammalian cells are selected from the group consisting of induced pluripotent stem cells, somatic cells, cardiac stem cells, and cardiosphere-derived cells.

62. The method of claim 61, wherein said mammalian cells are cardiosphere-derived cells.

63. The method of claim 61, wherein said cardiosphere-derived cells are present in a mammalian heart affected by a myocardial infarction.

64. The method of claim 53, wherein said contacting is ex vivo.

65. The method of claim 61, wherein said induced pluripotent stem cells are generated without the use of a virus by a method comprising contacting a differentiated mammalian cell with transducible polypeptides, wherein the transducible polypeptides comprise:
a) an Oct ¾ polypeptide linked to a cell penetration peptide,
a Sox2 polypeptide linked to a cell penetration peptide,
a NANOG polypeptide linked to a cell penetration peptide, and
a Lin28 polypeptide linked to a cell penetration peptide; or
b) an Oct ¾ polypeptide linked to a cell penetration peptide,
a Sox2 polypeptide linked to a cell penetration peptide, and
a Klf4 polypeptide linked to a cell penetration peptide,
wherein the cell penetration peptide comprises an amino terminus and a carboxy terminus, thereby generating an induced pluripotent stem cell.

66. The method of claim 65, wherein said induced pluripotent stem cell exhibits at least one characteristic of pluripotency if the induced pluripotent stem cell expresses of at least one human embryonic stem cell marker, has the ability to differentiate into greater than one cell type, has telomerase activity, or has the ability to divide 10-40 times.

67. A method for the repair of damaged or diseased cardiac tissue, comprising:
contacting said damaged or diseased cardiac tissue with a plurality of cells that exhibit at least one characteristic of cardiac differentiation,
wherein said plurality of cells were induced to exhibit said at least one characteristic of cardiac differentiation by a method comprising:
contacting said plurality of cells with a transducible peptide comprising an ISL1 polypeptide linked to a cell penetration peptide,
wherein said plurality of cells exhibits at least one characteristic of cardiac differentiation if the plurality of cells:
a) expresses at least one marker of cardiac differentiation selected from the group consisting of alpha-myosin heavy chain protein, natriuretic precursor A (ANP), ryanodine receptor, and SERCA; forms sarcomeres in culture,
b) appears visibly to be visibly beating, or
c) demonstrates spontaneous membrane depolarization;
wherein said plurality of cells repopulates said damaged or diseased cardiac tissue, thereby repairing said damaged or diseased cardiac tissue.

68. The method of claim 67, wherein said contacting of said plurality of cells with said transducible peptide is ex vivo.

69. The method of claim 67, wherein said contacting of said damaged or diseased cardiac tissue with said plurality of cells that exhibit at least one characteristic of cardiac differentiation is in vivo.

70. The method of claim 69, wherein said plurality of cells is positioned on a solid support prior to said contacting.

71. The method of claim 70, wherein said solid support comprises a synthetic or previously decellularized matrix.

72. The method of claim 67, wherein said plurality of cells comprises cardiosphere-derived cells.

73. An isolated mammalian cell that exhibits at least one characteristic of cardiac differentiation, wherein the isolated mammalian cell is generated by the method of claim 53.


SEQUENCE LISTING (41 sequences)

SEQ ID NO: 1 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgacgcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 2 - DNA, 1155 bp, Homo sapiens
catgagtcagtgaacagggaatgggtgaatgacatttgtgggtaggttatttctagaagttaggtgggcagcttggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 3 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 4 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 5 - DNA, 2518 bp, Homo sapiens
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 6 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 7 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 8 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 9 - DNA, 4014 bp, Homo sapiens
gtgcgggggaagatgtagcagcttcttctccgaaccaaccctttgccttcggacttctccggggccagcagccgcccgaccaggggcccggggccacgggctcagccgacgaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgagccacaatgggtgggggctattcttttgctatcaggaagttttgaggagcaggcagagtggagaaagtgggaatagggtgcattggggctagttggcactgccatgtatctcaggcttgggttcacaccatcaccctttcttccctctaggtggggggaaagggtgagtcaaaggaactccaaccatgctctgtccaaatgcaagtgagggttctgggggcaaccaggaggggggaatcaccctacaacctgcatactttgagtctccatccccagaatttccagcttttgaaagtggcctggatagggaagttgttttccttttaaagaaggatatataataattcccatgccagagtgaaatgattaagtataagaccagattcatggagccaagccactacattctgtggaaggagatctctcaggagtaagcattgtttttttttcacatcttgtatcctcatacccacttttgggatagggtgctggcagctgtcccaagcaatgggtaatgatgatggcaaaaagggtgtttgggggaacagctgcagacctgctgctctatgctcacccccgccccattctgggccaatgtgattttatttatttgctcccttggatactgcaccttgggtcccactttctccaggatgccaactgcactagctgtgtgcgaatgacgtatcttgtgcattttaactttttttccttaatataaatattctggttttgtatttttgtatattttaatctaaggccctcatttcctgcactgtgttctcaggtacatgagcaatctcagggatagccagcagcagctccaggtctgcgcagcaggaattactttttgttgtttttgccaccgtggagagcaactatttggagtgcacagcctattgaactacctcatttttgccaataagagctggcttttctgccatagtgtcctcttgaaaccccctctgccttgaaaatgttttatgggagactaggttttaactgggtggccccatgacttgattgccttctactggaagattgggaattagtctaaacaggaaatggtggtacacagaggctaggagaggctgggcccggtgaaaaggccagagagcaagccaagattaggtgagggttgtctaatcctatggcacaggacgtgctttacatctccagatctgttcttcaccagattaggttaggcctaccatgtgccacagggtgtgtgtgtgtttgtaaaactagagttgctaaggataagtttaaagaccaatacccctgtacttaatcctgtgctgtcgagggatggatatatgaagtaaggtgagatccttaacctttcaaaattttcgggttccagggagacacacaagcgagggttttgtggtgcctggagcctgtgtcctgccctgctacagtagtgattaatagtgtcatggtagctaaaggagaaaaagggggtttcgtttacacgctgtgagatcaccgcaaacctaccttactgtgttgaaacgggacaaatgcaatagaacgcattgggtggtgtgtgtctgatcctgggttcttgtctcccctaaatgctgccccccaagttactgtatttgtctgggctttgtaggacttcactacgttgattgctaggtggcctagtttgtgtaaatataatgtattggtctttctccgtgttctttgggggttttgtttacaaacttctttttgtattgagagaaaaatagccaaagcatctttgacagaaggttctgcaccaggcaaaaagatctgaaacattagtttggggggccctcttcttaaagtggggatcttgaaccatcctttcttttgtattccccttcccctattacctattagaccagatcttctgtcctaaaaacttgtcttctaccctgccctcttttctgttcacccccaaaagaaaacttacacacccacacacatacacatttcatgcttggagtgtctccacaactcttaaatgatgtatgcaaaaatactgaagctaggaaaaccctccatcccttgttcccaacctcctaagtcaagaccattaccatttctttctttcttttttttttttttttaaaatggagtctcactgtgtcacccaggctggagtgcagtggcatgatcggctcactgcagcctctgcctcttgggttcaagtgattctcctgcctcagcctcctgagtagctgggatttcaggcacccgccacactcagctaatttttgtatttttagtagagacggggtttcaccatgttgtccaggctggtctggaactcctgacctcaggtgatctgcccaccttggcttcccaaagtgctgggattacaggcatgagccaccatgctgggccaaccatttcttggtgtattcatgccaaacacttaagacactgctgtagcccaggcgcggtggctcacacctgtaatcccagcactttggaaggctgaggcgggcggatcacaaggtcacgagttcaaaactatcctggccaacacagtgaaaccccgtctctactaaaatacaaaaaaattagccgggtgtggtggtgcatgcctttagtcctagctattcaggaggctgaggcaggggaatcgcttgaacccgagaggcagaggttgcagtgagctgagatcgcaccactgcactccagcctggttacagagcaagactctgtctcaaacaaaacaaaacaaaacaaaaacacactactgtattttggatggatcaaacctccttaattttaatttctaatcctaaagtaaagagatgcaattgggggccttccatgtagaaagtggggtcaggaggccaagaaagggaatatgaatgtatatccaagtcactcaggaacttttatgcaggtgctagaaactttatgtcaaagtggccacaagattgtttaataggagacgaacgaatgtaactccatgtttactgctaaaaaccaaagctttgtgtaaaatcttgaatttatggggcgggagggtaggaaagcctgtacctgtctgtttttttcctgatccttttccctcattcctgaactgcaggagactgagcccctttgggctttggtgaccccatcactggggtgtgtttatttgatggttgattttgctgtactgggtacttcctttcccattttctaatcattttttaacacaagctgactcttcccttcccttctcctttccctgggaaaatacaatgaataaataaagacttattggtacgcaaactgtca

SEQ ID NO: 10 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 11 - DNA, 2377 bp, Homo sapiens
acccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaaaaaaaaaaaaaaaaaaaa

SEQ ID NO: 12 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 13 - DNA, 3057 bp, Homo sapiens
agttccccggccaagagagcgagcgcggctccgggcgcgcggggagcagaggcggtggcgggcggcggcggcacccggagccgccgagtgcccctccccgcccctccagccccccacccagcaacccgcccgtgacccgcgcccatggccgcgcgcacccggcacagtccccaggactccgcaccccgcgccaccgcccagctcgcagttccgcgccaccgcggccattctcacctggcggcgccgcccgcccaccgcccggaccacagcccccgcgccgccgacagccacagtggccgcgacaacggtgggggacactgctgagtccaagagcgtgcagcctggccatcggacctacttatctgccttgctgattgtctatttttataagagtttacaacttttctaagaatttttgtatacaaaggaacttttttaaagacatcgccggtttatattgaatccaaagaagaaggatctcgggcaatctgggggttttggtttgaggttttgtttctaaagtttttaatcttcgttgactttggggctcaggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcagtgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaaatcccacgtagtggatgtgacccacactgccaggagagagagttcagtatttttttttctaacctttcacactgtcttcccacgaggggaggagcccagctggcaagcgctacaatcatggtcaagttcccagcaagtcagcttgtgaatggataatcaggagaaaggaagagttcaagagacaaaacagaaatactaaaaacaaacaaacaaaaaaacaaacaaaaaaaacaagaaaaaaaaatcacagaacagatggggtctgatactggatggatcttctatcattccaataccaaatccaacttgaacatgcccggacttacaaaatgccaaggggtgactggaagtttgtggatatcagggtatacactaaatcagtgagcttggggggagggaagaccaggattcccttgaattgtgtttcgatgatgcaatacacacgtaaagatcaccttgtatgctctttgccttcttaaaaaaaaaaaaagccattattgtgtcggaggaagaggaagcgattcaggtacagaacatgttctaacagcctaaatgatggtgcttggtgagtcgtggttctaaaggtaccaaacgggggagccaaagttctccaactgctgcatacttttgacaaggaaaatctagttttgtcttccgatctacattgatgacctaagccaggtaaataagcctggtttatttctgtaacatttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaagcaaacttctattttgtatatttgtaaactacaaagtaaaaaaaaatgaacattttgtggagtttgtattttgcatactcaaggtgagaaataagttttaaataaacctataatattttatctgaacgacaaaaaaaaaaaaaaa

SEQ ID NO: 14 - protein, 483 aa, Homo sapiens
MRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLGAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 15 - protein, 301 aa, human herpesvirus 1
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTQRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 16 - protein, 143 aa, human herpesvirus 1
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 17 - protein, 5 aa, human herpesvirus 1
RSASR

SEQ ID NO: 18 - protein, 5 aa, human herpesvirus 1
RTASR

SEQ ID NO: 19 - protein, 5 aa, human herpesvirus 1
RSRAR

SEQ ID NO: 20 - protein, 5 aa, human herpesvirus 1
RTRAR

SEQ ID NO: 21 - protein, 5 aa, human herpesvirus 1
ATATR

SEQ ID NO: 22 - protein, 6 aa, human herpesvirus 1
RSAASR

SEQ ID NO: 23 - protein, 11 aa, human herpesvirus 1
YGRKKRRQRRR

SEQ ID NO: 24 - protein, 16 aa, Drosophila melanogaster
RQIKIWFQNRRMKWKK

SEQ ID NO: 25 - protein, 561 aa, Artificial Sequence
  VP22-0ct3/4 fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNKGNSADIQHSGGRSSLEGPRFEQKLISEEDLNMHTGHHHHHH

SEQ ID NO: 26 - protein, 573 aa, Artificial Sequence
  Oct3/4-VP22 fusion polypeptide
MASMTGGQQMGRDLGTAGALFKGSTSPVWWNCPXMAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 27 - protein, 518 aa, Artificial Sequence
  VP-22-Sox2 fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMKGNSADIQHSGGRSSLEGPRFEQKLISEEDLNMHTGHHHHHH

SEQ ID NO: 28 - protein, 530 aa, Artificial Sequence
  Sox2-VP22 fusion polypeptide
MASMTGGQQMGRDLGTAGALFKGSTSPVWWNCPXMYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 29 - protein, 506 aa, Artificial Sequence
  VP-22-Nanog fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVKGNSADIQHSGGRSSLEGPRFEQKLISEEDLNMHTGHHHHHH

SEQ ID NO: 30 - protein, 518 aa, Artificial Sequence
  Nanog-VP22 fusion polypeptide
MASMTGGQQMGRDLGTAGALFKGSTSPVWWNCPXMSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 31 - protein, 410 aa, Artificial Sequence
  VP-22-Lin28 fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNKGNSADIQHSGGRSSLEGPRFEQKLISEEDLNMHTGHHHHHH

SEQ ID NO: 32 - protein, 422 aa, Artificial Sequence
  Lin28-VP22 fusion polypeptide
MASMTGGQQMGRDLGTAGALFKGSTSPVWWNCPXMGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 33 - protein, 655 aa, Artificial Sequence
  VP-22-c-myc fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAKGNSADIQHSGGRSSLEGPRFEQKLISEEDLNMHTGHHHHHH

SEQ ID NO: 34 - protein, 667 aa, Artificial Sequence
  c-myc-VP22 fusion polypeptide
MASMTGGQQMGRDLGTAGALFKGSTSPVWWNCPXMDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCAKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 35 - protein, 684 aa, Artificial Sequence
  VP-22-Klf4 fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFKGNSADIQHSGGRSSLEGPRFEQKLISEEDLNMHTGHHHHHH

SEQ ID NO: 36 - protein, 696 aa, Artificial Sequence
  Klf4-VP22 fusion polypeptide
MASMTGGQQMGRDLGTAGALFKGSTSPVWWNCPXMRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 37 - DNA, 1050 bp, Homo sapiens
atgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatga

SEQ ID NO: 38 - protein, 349 aa, Homo sapiens
MGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEA

SEQ ID NO: 39 - protein, 550 aa, Artificial Sequence
  VP-22-Isll fusion polypeptide
STAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEGTELGSTSPVWWNCPXMGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEAKGNSADIQHSGGRSSLEGPRFEQKLISEEDLNMHTGHHHHHH

SEQ ID NO: 40 - protein, 562 aa, Artificial Sequence
  Isll-VP22 fusion polypeptide
MASMTGGQQMGRDLGTAGALFKGSTSPVWWNCPXMGDMGDPPKKKRLISLCVGCGNQIHDQYILRVSPDLEWHAACLKCAECNQYLDESCTCFVRDGKTYCKRDYIRLYGIKCAKCSIGFSKNDFVMRARSKVYHIECFRCVACSRQLIPGDEFALREDGLFCRADHDVVERASLGAGDPLSPLHPARPLQMAAEPISARQPALRPHVHKQPEKTTRVRTVLNEKQLHTLRTCYAANPRPDALMKEQLVEMTGLSPRVIRVWFQNKRCKDKKRSIMMKQLQQQQPNDKTNIQGMTGTPMVAASPERHDGGLQANPVEVQSYQPPWKVLSDFALQSDIDQPAFQQLVNFSEGGPGSNSTGSEVASMSSQLPDTPNSMVASPIEAKGNSADIQHSGGRPSSTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVEFEQKLISEEDNMHTGHHHHHH

SEQ ID NO: 41 - protein, 7 aa, Simian virus 40
PKKKRKV